(19) United States
(12) Patent Application Publication
(10) Pub. No.: US 2012/0052581 A1
(43) Pub. Date: Mar. 1, 2012

(54) Microporous Cell Culture Substrates

(76) Inventors: Michael Edward DeRosa, Painted Post, NY (US); Hongwei Hanna Rao, Horseheads, NY (US)

(21) Appl. No.: 13/218,076

(22) Filed: Aug. 25, 2011

Related U.S. Application Data
(60) Provisional application No. 61/377,640, filed on Aug. 27, 2010.

(51) Int. Cl. C12N 5/02; B29C 44/22

(52) U.S. Cl. 435/396; 264/48

(57) ABSTRACT

Surfaces of thermoplastic articles are rendered microporous by contacting the surface with a composition that includes a solvent. The article has a birefringence of 0.0001 or greater and the composition has a solvent strength configured to swell but not dissolve the polymer.

### CROSS-REFERENCE TO RELATED APPLICATIONS

This application claims the benefit of priority under 35 U.S.C. §119 of U.S. Provisional Application Ser. No. 61/377,640 filed on Aug. 27, 2010 the content of which is relied upon and incorporated herein by reference in its entirety.

### FIELD

The present disclosure relates to microporous thermoplastic articles and methods for making surfaces of thermoplastic articles, or portions thereof, microporous.

### BACKGROUND

Porous polymeric materials have been made commercially for many decades for a large number of applications, and there are numerous methods of making such materials. For example, thermoplastic foams can be made by forming a polymer melt or solution with the addition of a foaming agent to generate gas bubbles with the addition of heat or release of pressure. Porous polymers have also been made by template leaching in which a solid pore former, such as salt or sugar particles, is added to a polymer. The pore former can be leached out later with a solvent leaving behind a porous structure. Some solid pore formers can be heated in a reaction that transforms the pore former into a gas leaving behind a pore. Microstructured open cell membranes have been made for many years by using thermally induced phase separation or non-solvent induced phase separation processes. Porous membranes can also easily be made by sintering polymer particles of controlled size. Porous films can be made by stretching extruded films transverse to the drawing direction to open up elongated pores. Track etching can also be used in which a polymer film is bombarded with radiation and then subsequently etched in a solvent to reveal straight pores having a narrow size distribution. All of the aforementioned methods of making porous polymers enable one to make polymer articles in which pores reside throughout the entire volume of the part or through the entire thickness of a film.

Some methods are capable of making just the surface of the polymer porous. One common method of making asymmetric membranes involves using a modified non-solvent induced phase separation process. In this method, a polymer solution is cast to make a film. The film is then placed in a bath before the solvent fully evaporates. The bath has a liquid that is miscible with the solvent but is a poor solvent for the polymer. As the non-solvent diffuses into the film, phase separation occurs and creates microporous domains. The result is a film that is dense on one side, i.e. little or no porosity, and has a gradient of pore sizes across the thickness of the remainder of the film.

Solvent induced crazing is another method of introducing openings into a thermoplastic surface. Certain solvents cause microcracks and or crazes in glassy thermoplastics. When a glassy thermoplastic is placed under an external load it becomes more susceptible to solvent attack. This is typically referred to as environmental stress cracking or crazing (ESC). ESC is undesirable because it makes molded polymer objects weaker over time. Eventually ESC often leads to catastrophic long term mechanical failure. Injection molded plastics that have residual molding stress also are more susceptible to ESC. Residual molding stress can cause other mechanical and optical problems in transparent thermoplastic polymers in addition to ESC. Therefore, it is usually undesirable to make injection molded thermoplastic parts with high residual stress and great effort is taken to make parts that are as stress free as possible.

### BRIEF SUMMARY

The present disclosure describes, among other things, a method of fabricating a microporous surface on thermally formed glassy amorphous thermoplastic articles. The thermoplastic articles are formed so as to have at least a certain amount of molecular orientation. This can be done, for example, by making the parts with residual molding stress. Such residual stress can be identified by birefringence, which should exceed a minimum value. The articles are then treated with a liquid composition comprising a solvent having a proper solubility strength to swell but not dissolve the polymer. As the polymer swells, micropores are created at the surface of the polymer. The micropores created under these conditions resemble pores created by phase separation techniques and do not resemble microcracks or crazes. The process and method described here may be used on nearly any glassy amorphous thermoplastics, such as polystyrene, polymethylmethacrylate or other acrylic polymers, cyclic olefin copolymer, or styrene maleic anhydride.

In various embodiments described herein, a method for fabricating microporous surface on a thermally formed glassy amorphous thermoplastic article includes (i) contacting a surface of the article with a composition comprising a solvent for the thermoplastic article, wherein the composition has a solubility strength configured to cause swelling of the thermoplastic article without dissolving the thermoplastic article, and (ii) removing the composition from the thermoplastic article. The article has a birefringence of 0.0001 or greater.

In many embodiments, a method for forming a microporous cell culture substrate includes: (i) molding a non-porous cell culture substrate from a thermoplastic polymer such that the non-porous substrate has a birefringence of 0.0001 or greater; (ii) contacting a surface of the non-porous substrate with a composition comprising a solvent for the thermoplastic polymer, wherein the composition is configured to cause swelling of the thermoplastic polymer without dissolving the thermoplastic polymer; and (iii) removing the composition from the surface to yield a cell culture substrate having a microporous region contiguous with the surface.

In numerous embodiments described herein, a cell culture article has a surface for culturing cells. The surface consists essentially of a molded polymeric material having a surface for culturing cells. The surface comprises a microporous structure formed from the polymeric material.

In various embodiments, a method for fabricating microporous surface on a polystyrene article includes: (i) providing a polystyrene article having a birefringence of 0.0001 or greater; (ii) contacting a surface of the article with a composition comprising a solvent for the polystyrene, wherein the composition is configured to cause swelling of the polystyrene article without dissolving the polystyrene article; and (iii) removing the composition from the polystyrene article.

In some embodiments, a method for fabricating microporous surface on a cyclic olefin copolymer article includes: (i) providing a cyclic olefin copolymer article having a birefringence of 0.0001 or greater; (ii) contacting a surface of the article with a composition comprising a solvent for the cyclic olefin copolymer, wherein the composition is configured to cause swelling of the cyclic olefin copolymer article without dissolving the cyclic olefin copolymer article; and (iii) removing the composition from the cyclic olefin copolymer article.

In numerous embodiments described herein, a cell culture article has a microporous substrate for culturing cells, wherein the substrate comprises an open cell microporous structure having an average pore size of 50 micrometers or greater. The pores may be made from a non-porous thermoplastic substrate by contacting the non-porous substrate with a solvent/non-solvent mixture. In many embodiments, the non-solvent is water.

In many embodiments, a method for forming a microporous cell culture substrate, wherein the substrate allows cells cultured on the articles to be viewed by routine light microscope techniques, includes: (i) providing a thermally formed non-porous thermoplastic cell culture substrate having a birefringence of 0.0001 or greater; (ii) contacting a surface of the non-porous substrate with a composition comprising a non-solvent and a solvent for the thermoplastic polymer, wherein the composition is configured to cause swelling of the thermoplastic polymer without dissolving the thermoplastic polymer, wherein the non-solvent is water; and (iii) removing the composition from the surface to yield a cell culture substrate having a microporous region contiguous with the surface, wherein the resulting microporous region has an average pore size of 50 micrometers or greater.

The devices, articles and methods described herein may provide one or more advantages over prior thermoplastic articles having microporous structure or methods for making such articles. For example, embodiments of the methods described herein allow for creation of a porous surface on a molded bulk thermoplastic article, whereas most conventional methods of making porous polymer parts create porous articles that have porosity throughout the entire body or thickness. Embodiments of the methods described herein can be applied to existing molded thermoplastic products, as they may be performed as a post-processing step, and can be easily integrated with existing production thermoplastic molding methods and equipment requiring little to no modification of currently used molding set-ups. Further, a post-molding step can be a relatively inexpensive means of adding surface porosity to molded thermoplastic articles. In many cases, the pore size can be controlled moderately by simple choice of solvent mixture. Simple and inexpensive stenciling processes can be used to make patterns with the desired amount of surface porosity on molded thermoplastic articles using the processes described herein. In addition, unlike embodiments of the methods described herein, existing thermoplastic molding or hot embossing processes do not allow for making three dimensional pore structures with interconnected surface pores or complex pore shapes in one step. These and other advantages of the various embodiments of the devices and methods described herein will be readily apparent to those of skill in the art upon reading the disclosure presented herein.

### BRIEF DESCRIPTION OF THE DRAWINGS

FIG. 1 is a schematic diagram of a Hansen solubility sphere of a polymer and shows representative coordinates of a test solvent or mixture.

FIG. 2 is a schematic diagram of a Hansen solubility sphere showing coordinates relative to the sphere of solvents capable of causing micropore formation.

FIGS. 3-4 are flow diagrams of embodiments of methods for generating a microporous network from a thermoplastic article.

FIG. 5 is a schematic, diagrammatic depiction of a method for generating a patterned microporous network on a surface of a thermoplastic article.

FIGS. 6A-D are images showing stress birefringence patterns in polystyrene (A) and cyclic olefin copolymer—Topas® 8007-X10 (C) multi-well plates and images of the multi-well plates after solvent treatment in accordance with the teachings presented herein: (B) polystyrene; (D) TOPAS.

FIGS. 7A-C are images showing stress birefringence patterns (A) in a center gate molded polystyrene 685D plate, the plate after solvent treatment in accordance with the teachings presented herein (B), and a flow simulation result showing the injection pressure plot from which the shear field can be inferred (C).

FIGS. 8A-E are images of polystyrene Dow Styron® 685D surfaces treated with different volume/volume fractions of tetrahydrofuran/isopropanol: 25/75 (A); 35/65 (B); 40/60 (C); 50/50 (D); and 60/40 (E).

FIGS. 9A-B are images of a polystyrene 6-well plate where each well bottom was treated for 30 seconds with a 40/60 mixture of tetrahydrofuran/isopropanol in accordance with the teachings presented herein. FIG. 9B is a magnified view of the area indicated in FIG. 9A.

FIGS. 10A-B are 75× magnified images of well bottoms of molded polystyrene 6-well plates that were treated with a 40/60 mixture of tetrahydrofuran/isopropanol (A) and a mixture of 50/50 tetrahydrofuran/water (B).

FIG. 11 presents images at different magnifications of a cyclic olefin copolymer (TOPAS 8007-X10) molded 96-well insert plate patterned with a 30 second methylene chloride dip process in accordance with the teachings presented herein to produce microporous surfaces corresponding to the well bottoms of plate.

FIGS. 12A-B are images of polystyrene film patterned with a tetrahydrofuran (THF)/isopropanol (IPA) solvent mixture (40/60 v/v %) for 20 seconds at room temperature. One half of the patterned polystyrene film was exposed to oxygen plasma at 30 W at 60 s while the other half was protected. The dotted line depicts the boundary between the two sides. (A) A transparent self-adhesive tape was adhered across the oxygen plasma treated and untreated sides and a droplet of red colored food dye was separately pipetted onto the two sides. (B) 90 days after oxygen plasma treatment, a droplet of water was separately pipetted onto the treated and untreated sides.

FIGS. 13A-B are backscattering SEM images of well bottoms similar to those depicted in FIG. 10B and FIG. 10A, respectively.

FIG. 14 is an optical microscope bright field image of a well of a cyclic olefin copolymer (TOPAS) cell culture article treated with 95/5 v/v % tetrahydrofuran/water.

FIGS. 15A-B are light microscope images of cells cultured on a polystyrene substrate rendered microporous with tetrahydrofuran/water (A) and tetrahydrofuran/isopropanol (B).

FIGS. 16A-B are fluorescent images of stained human mesenchymal cells cultured on a polystyrene substrate rendered microporous with tetrahydrofuran/water (A) and tetrahydrofuran/isopropanol (B).

FIG. 17 is a bar graph showing results of a cell attachment assay of human mesenchymal stem cells cultured on a variety of substrates.

The schematic drawings presented herein are not necessarily to scale. Like numbers used in the figures refer to like components, steps and the like. However, it will be understood that the use of a number to refer to a component in a given figure is not intended to limit the component in another figure labeled with the same number. In addition, the use of different numbers to refer to components is not intended to indicate that the different numbered components cannot be the same or similar.

### DETAILED DESCRIPTION

In the following detailed description, reference is made to the accompanying drawings that form a part hereof, and in which are shown by way of illustration several specific embodiments of devices, systems and methods. It is to be understood that other embodiments are contemplated and may be made without departing from the scope or spirit of the present disclosure. The following detailed description, therefore, is not to be taken in a limiting sense.

All scientific and technical terms used herein have meanings commonly used in the art unless otherwise specified. The definitions provided herein are to facilitate understanding of certain terms used frequently herein and are not meant to limit the scope of the present disclosure.

As used in this specification and the appended claims, the singular forms “a”, “an”, and “the” encompass embodiments having plural referents, unless the content clearly dictates otherwise.

As used in this specification and the appended claims, the term “or” is generally employed in its sense including “and/or” unless the content clearly dictates otherwise.

As used herein, “have”, “having”, “include”, “including”, “comprise”, “comprising” or the like are used in their open ended sense, and generally mean “including, but not limited to.” It will be understood that the terms “consisting of” and “consisting essentially of” are subsumed in the term “comprising,” and the like. For example, a method for forming a microporous cell culture substrate that comprises (i) molding a non-porous cell culture substrate from a thermoplastic polymer such that the non-porous substrate has a birefringence of 0.0001 or greater; (ii) contacting a surface of the non-porous substrate with a composition comprising a solvent for the thermoplastic polymer, wherein the composition is configured to cause swelling of the thermoplastic polymer without dissolving the thermoplastic polymer; and (iii) removing the composition from the surface to yield a cell culture substrate having a microporous region contiguous with the surface may consist of, or consist essentially of, molding the substrate, contacting the surface with composition, and removing the composition.

“Consisting essentially of”, as it relates to a compositions, articles, systems, apparatuses or methods, means that the compositions, articles, systems, apparatuses or methods include only the recited components or steps of the compositions, articles, systems, apparatuses or methods and, optionally, other components or steps that do not materially affect the basic and novel properties of the compositions, articles, systems, apparatuses or methods.

Any direction referred to herein, such as “top,” “bottom,” “left,” “right,” “upper,” “lower,” “above,” below,” and other directions and orientations are described herein for clarity in reference to the figures and are not to be limiting of an actual device or system or use of the device or system. Many of the devices, articles or systems described herein may be used in a number of directions and orientations.

As used herein, “microporous structure” refers to a structure having pores or interstices of an average diametric size of less than 1000 micrometers.

As used herein, “pore” means a cavity or void in a surface, a body, or both a surface and a body of a solid article, where the cavity or void has at least one outer opening at a surface of the article.

As used herein, “interstice” means a cavity or void in a body of a solid polymer not having a direct outer opening at a surface of the article, i.e., not a pore, but may have an indirect outer opening or pathway to an outer surface of the article by way of one or more links or connections to adjacent or neighbor “pores” “interstices,” or a combination thereof.

As used herein, a “solvent” for a polymeric sheet is a composition capable of causing gelation, swelling or solubilization of at least a portion of the polymeric sheet when contacted with the sheet. A “non-solvent” for a polymeric sheet means a composition that does not cause gelation, swelling or solubilization of the polymeric sheet when contacted with the sheet.

The present disclosure describes, among other things, methods for forming microporous structures from surfaces of molded glassy amorphous thermoplastic articles. The articles are molding to increase the molecular orientation, which results in increased residual molding stress and birefringence. While this runs contrary to how most thermoplastic molding operations are run and is considered to be unconventional, this approach has been found important in order to create the desired surface porosity. The articles, or parts thereof, are then contacted with a composition comprising a solvent. The composition is capable of swelling the polymer but not dissolving the polymer. It has been found that of limited range of solubility strength is important for achieving a desired microporous structure. The solubility strength of a solvent composition may be appropriately adjusted with the addition of a non-solvent.

1. Formation of Thermoplastic Article

It has been found that, in order to produce a microporous surface from a thermoformed thermoplastic article, the article should be made with at least a minimum amount of molecular orientation. Without intending to be bound by theory, it is believed that this will allow the solvent to penetrate into the surface and create the microporous texture. The relative level of molecular orientation of a transparent thermoplastic can be determined by the degree of optical birefringence in the molded part. The birefringence (Δn) is defined in Equation 1:

Δn=n₁−n₂ (1)

where n₁ an n₂ are the refractive indices of light polarized parallel (n₁) and perpendicular (n₂) to the deformation or flow direction of the polymer during the forming process. When an oriented transparent molded polymer is placed between crossed polarizing sheets, the birefringence pattern can be seen with multiple colored fringes. The birefringence can be calculated from these fringes. Higher order fringes indicate higher level of birefringence and hence orientation.

The residual stress in a material is also related to the birefringence of the material as well. The birefringence is related to the stress (σ) by a constant called the stress optic coefficient (SOC) in Equation 2:

$$SOC = \frac{Δ n}{σ} \qquad ( 2 )$$

To achieve high birefringence, and hence molecular orientation, a polymer can be molded with high residual stress by controlling the molding parameters such as injection speed, melt temperature, gate location and size, mold temperature, etc.

For example, it has been found that the size and location of the injection gate into a mold cavity may be chosen to create a sufficiently high shear zone across the volume of the mold to increase molecular orientation. Similarly, increased injection speeds tend to result in increased shear stress and thus increased molecular orientation. By way of further example, the further below the glass transition temperature of a particular polymeric material that the setting of the mold temperature is held, the more the residual stress that may be molded into the article. For sheets or films, uni- or biaxial stretching results in molecular orientation. It will be understood that these are merely examples of how one can produce an article having sufficient molecular orientation to achieve a birefringence of greater than 0.0001. Other techniques may be used and are generally known in the art, including drawing, calendaring, blow molding, film blowing and the like.

Thermoplastic articles having sufficient residual stress or birefringence may be formed by any suitable method, such as extrusion, blow molding, injection molding or the like. The articles for use with the methods described herein preferably have a birefringence 0.0001 or greater, such as 0.001 or greater or 0.01 or greater.

The methods described herein may be applied to any suitable glassy amorphous thermoplastic polymer, such as polystyrenes, polymethylmethacrylates or other acrylic polymers, cyclic olefin copolymers, or styrene maleic anhydride polymers. The articles made from these polymers may be films or other extruded articles or molded articles.

2. Formation of Microporous Structure

Once the thermoplastic polymeric article is formed, e.g., molded or extruded, with sufficient residual stress; e.g., having a birefringence of 0.0001 or greater, the article, or a portion thereof, may be contacted with a composition comprising a solvent. The composition should have a solvent strength sufficient to swell but not dissolve the polymeric material; e.g. at room temperature. To achieve a solvent of a strength that will cause the polymer to swell, the solubility parameter is typically about the same or greater than that of the polymer. If the solvent strength is too great; e.g. it dissolves the polymer, the solvent composition may be appropriately adjusted with the addition of a non-solvent.

The composition comprising the solvent may include one or more solvents and one or more non-solvents. As generally understood in the art, different polymeric materials are soluble or swellable in different solvents. Accordingly, the one or more solvents employed will be dependent on the polymeric material of the article. Any solvent suitable for solubilizing or swelling a polymer of the article may be employed. Such solvents are generally known in the art. For example, for polystyrene, suitable solvents include tetrahydrofuran, methylethyl ketone, ethyl acetate, and acetone. For cyclic polyolefins suitable solvents include methylene chloride, and tetrahydrofuran. For styrene maleic anhydride polymeric sheets, suitable solvents include acetone, tetrahydrofuran, 1,3-dioxolane, methylethyl ketone, toluene, ethyl acetate, and N-methylpyrolidone. It will be understood that these are only a few examples of the suitable solvents that may be used for these polymers and that other solvents may readily be used and that other polymers with appropriate solvents may be used in accordance with the teachings herein to generate a microporous structure.

Any one or more non-solvents may be employed. As with solvents, some non-solvents may be selective to the polymeric article for which it is desirable to impart an microporous region. However, many non-solvents will work with most, if not all, polymers. By way of example, suitable non-solvents for polystyrene include water and an alcohol, such as a C1-C4 unsubstituted alcohol, which includes isopropanol, ethanol, and methanol. For cyclic polyolefins, suitable non-solvents include water and an alcohol, such as a C1-C4 unsubstituted alcohol. For styrene maleic anhydride polymers, suitable non-solvents include water and C1-C4 unsubstituted alcohols which include isopropanol, ethanol, and methanol. It will be understood that these are only a few examples of the suitable non-solvents that may be used for these polymers and that other non-solvents may readily be used and that other polymers with appropriate non-solvents may be used in accordance with the teachings herein to generate a microporous structure.

As indicated above, it has been found that the solubility strength of the composition comprising the one or more solvents should be finely controlled to produce a desired microporous structure. It will be understood that the ratio and composition of solvent and non-solvent will vary depending on a number of factors, including the composition of the polymeric article and the solubility of the polymeric article in the solvent employed. In some cases, no non-solvent is required to achieve a desired solubility parameter. In other cases, the non-solvent constitutes up to 70 percent or more of the volume of the composition comprising the one or more solvents.

By way of example, it has been found that solvent compositions having the following ratios, on a volume/volume basis, of solvent and non-solvent are suitable for forming microporous structures from polystyrene articles having a birefringence of 0.0001 or greater: tetrahydrofuran (THF)/isopropanol in range of 35/65-50/50; THF/ethanol in a range of 35/65-50/50; ethyl acetate/isopropanol in a range of 45/55-65/35; and THF/water in a range of 40/60-70/30, such as 45/55-65/35. By way of further example, it has been found that solvent compositions having the following ratios, on a volume/volume basis, of solvent and non-solvent are suitable for forming microporous structures from cyclic olefin copolymer articles having a birefringence of 0.0001 or greater: methylene chloride (single solvent); THF/isopropanol in a range of 75/25-90/10; and THF/water in a range of 80/20-98/2, such as 90/10-95/5. It will be understood that these are just examples that were found to work successfully and these do not constitute an exhaustive list of solvents, non-solvents, and polymers for which the processes described herein will produce microporous surface structures.

To the extent that the ranges of ratios of solvent and non-solvent may vary from polymeric article to polymeric article and from solvent to solvent; a suitable range may be readily identified by those of skill in the art. For example, (i) one may try a variety of ratios of known solvents and non-solvents for a particular polymer to determine whether the ratio is suitable for forming a porous structure from the article, (ii) identify those ratios that are suitable and expand around those ratios to find the boundaries of suitable ranges. Any suitable test or assay may be employed to determine whether the composition comprising solvent and non-solvent is capable of imparting a microporous structure to at least portion of the polymeric article may be performed. For example, microscopic examination of article after contact and removal of the solvent/non-solvent composition may be used to identify whether suitable porous regions have formed.

Alternatively or additionally, a strength of a solvent or solvent mixture that is suitable for inducing pore formation on a polymeric article may be determined using Hansen solubility parameters (see, e.g., Hansen, C. M., Hansen Solubility Parameters a User's Handbook 2nd Ed., CRC Press, Boca Raton, 2007). We have found that solvent or solvent mixtures that have Hansen Relative Energy Difference (RED) values in a range of the polymer solubility boundary have been found to cause microporous formation on molded thermoplastic articles. In particular, fluid compositions comprising one or more solvents, which may also contain one or more non-solvents, that have a RED of between about 0.5 and about 2 may be suitable for forming microporous structures on polymeric articles. Preferably, the fluid composition has a RED of between about 0.75 and about 1.6, such as between about 0.8 and about 1.5 or between about 0.85 and about 1.45.

A more detailed discussion of Hansen solubility parameters and RED is discussed in co-pending U.S. patent application Ser. No. 13/217,818, entitled MICROPOROUS THERMOPLASTIC SHEETS, having attorney docket no. SP11-197, naming Michael DeRosa, Todd Upton, and Ying Zhang as inventors, and filed on the same date herewith, which application is hereby incorporated herein by reference in its entirety to the extent that it does not conflict with the disclosure presented herein.

According to Hansen, the total cohesion energy (E) of a liquid is defined by the energy required to convert a liquid to a gas. This can be experimentally measured by the heat of vaporization. Hansen described the total cohesion energy as being comprised of three primary intermolecular forces: atomic dispersion forces (E_{D}), molecular permanent dipole-dipole interactions (E_{P}), and molecular hydrogen bonding interactions (E_{H}). When the cohesion energy is divided by the molar volume (V) the total cohesive energy density of the liquid is given by Equation 3:

E/V=E_{D}/V+E_{P}/V+E_{H}/V. (3)

The solubility parameter (δ) of the liquid is related to the cohesive energy density as shown in Equation 4:

δ=(E/V)^{1/2} (4)

where δ is the Hildebrand solubility parameter. The three Hansen solubility components of a liquid are thus given in Equation 5:

δ²=δ_{D}²+δ_{P}²+δ_{H}² (5)

These three parameters have been tabulated for thousands of solvents and can be used to describe polymer-solvent interactions (see, e.g., Hansen, 2007).

Solubility parameters exist for solid polymers as well as liquid solvents (see, e.g., Hansen, 2007). Polymer-solvent interactions are determined by comparing the Hansen solubility parameters of the polymer to that of a solvent or solvent mixture defined by the term Rₐ as shown in Equation 6:

Rₐ²=4(δ_{D2}−δ_{D1})²+(δ_{P2}−δ_{P1})²+(δ_{H2}−δ_{H1})² (6)

where subscripts 1 and 2 refer to the solvent or solvent mixture and polymer respectively. Rₐ is the distance in three dimensional space between the Hansen solubility parameters of a polymer and that of a solvent. A “good” solvent for a particular polymer has a small value of Rₐ. This means the solubility parameters of the polymer and solvent are closely matched and the solvent will quickly dissolve the polymer. Rₐ will increase as a solvent's Hansen solubility parameters become more dissimilar to that of the polymer.

The solubility of a particular polymer is not technically described by just the three parameters in Equation (3). A good solvent does not have to have parameters that perfectly match that of the polymer. There is a range of solvents that will work to dissolve the polymer. The Hansen solubility parameters of a polymer are defined by δ_{D}, δ_{P}, and δ_{H} which are the coordinates of the center of a solubility sphere which has a radius (Rₒ). Rₒ defines the maximum distance from the center of the sphere that a solvent can be and still dissolve the polymer.

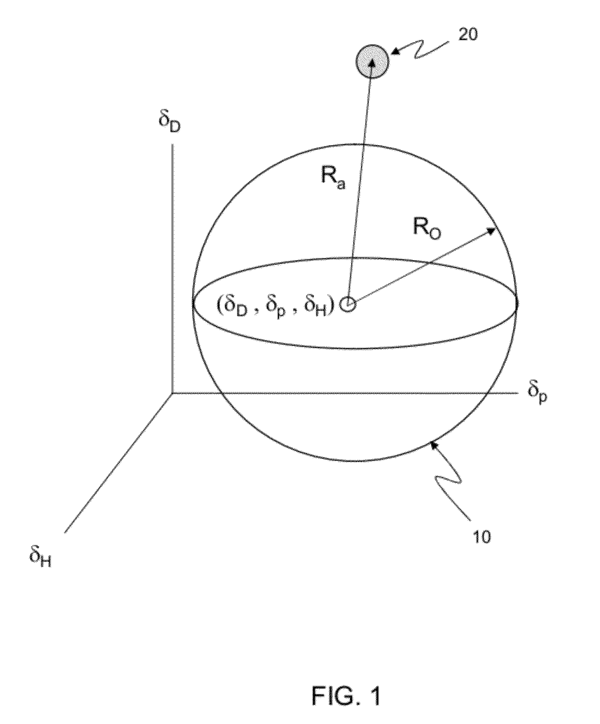

A schematic of a polymer solubility sphere 10 and a test solvent or mixture coordinates 20 are shown in FIG. 1, where the sphere 10 defined by its center coordinates (δ_{D}, δ_{P}, δ_{H}) and a radius Rₒ. Solvents that lie within the sphere 10 will dissolve the polymer. The coordinates 20 of an example of a test solvent or solvent mixture, which are at a distance, Rₐ, from the center of the solubility sphere, are also depicted in FIG. 1.

The strength of a solvent for a polymer is determined by comparing Rₐ to Rₒ. A term called the Relative Energy Difference (RED) is given by Equation 7:

RED=Rₐ/Rₒ. (7)

Using RED values is a simple way to evaluate how “good” a solvent will be for a given polymer. Solvents or solvent mixtures that have a RED number much less than 1 will have Hansen solubility parameters close to that of the polymer and will dissolve the polymer quickly and easily. Liquids that have RED numbers much greater than 1 will have Hansen solubility parameters further away from the polymer and will have little or no effect on the polymer. Liquids that have RED numbers close to one will be on the boundary between good and poor solvents. These liquids usually swell the polymer and belong to a class of solvents that typically cause environmental stress cracking and crazing (see, e.g., Hansen, C. M.; Just, L., “Prediction of Environmental Stress Cracking in Plastics with Hansen Solubility Parameters, Ind. Eng. Chem. Res., 40, 21-25, 2001).

It will be understood that the width of suitable RED value ranges for inducing pore formation depend on the amount of residual stress in the polymer article, with higher residual stress resulting in higher RED values. That is, the higher the amount of residual stress, or birefringence, the larger the RED value will be for the upper boundary. Polymeric articles that have lower stress or birefringence will require solvents or solvent mixtures that are closer to the center of the sphere within the shaded region to produce porous surfaces.

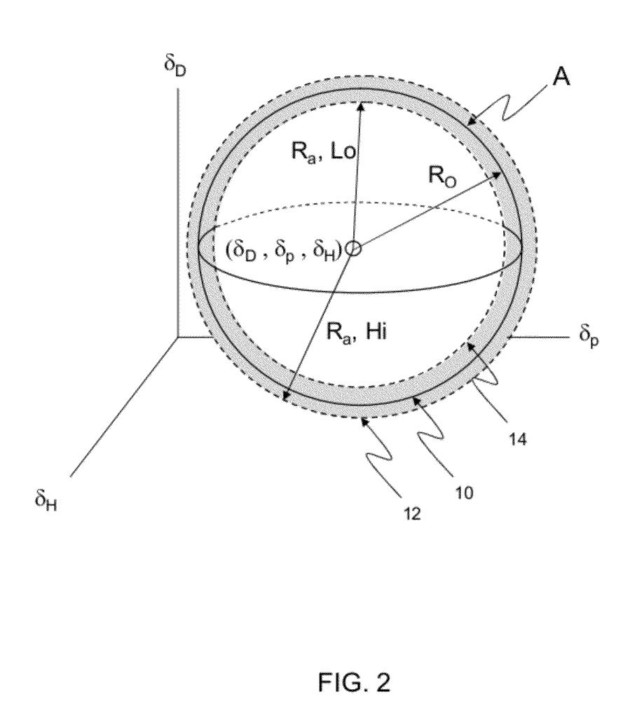

FIG. 2 is a schematic of illustration fluid compositions having suitable solubility parameters to form microporous surfaces. The polymer solubility sphere is defined by its center coordinates (δ_{D}, δ_{P}, δ_{H}) and a radius Rₒ. Solvent and solvent mixtures that will form microporous surfaces will have solubility parameters that reside in a range around the polymer solubility sphere, as indicated by the shaded area (A) in FIG. 2. The outer boundary of solubility parameters that are suitable for forming the microporous surfaces is depicted in FIG. 2 as being defined by the radius Rₐ, Hi (12) and defines the upper RED value. The lower boundary of solubility parameters that are suitable for forming the microporous surfaces is depicted in FIG. 2 as being defined by the radius Rₐ, Lo (14) and defines the lower RED value. A similar framework for environmental stress crazing was discussed by Hansen (see, Hansen, 2007 and Hansen, 2001). Hansen used this for the purpose of describing mechanical reliability of polymers and treated stress crazing as phenomenon to be avoided. Here we are using this range of polymer/solvent interactions to define the desirable characteristics for producing microporous surfaces.

It will also be understood that the values of R₀ value of a given polymer may change depending on the amount of residual stress or birefringence of the article. The value obtained for R₀ may also change based on the solvents or non-solvents used to determine the R₀ value. If solvents or combinations of solvents and non-solvents are used that are within the micropore forming range (e.g. shaded area of the sphere in FIG. 2), then the value of R₀ may more readily change depending on residual stress or birefringence. However, if solvents or combinations of solvents and non-solvents are used that are not within the micropore forming range, the determined R₀ value may not change with changing residual stress or birefringence values. The depth that the generated microporous structure may extend through the article may vary and may be controlled by controlling solvent contact time, temperature, and the like. For example, the microporous region may be formed only on the surface of the article, having a depth of about, e.g., 10 micrometers to about 100 micrometers, or may extend through the entire depth of the article, depending on the conditions used. The thickness of the non-porous starting article will also affect the extent to which the microporous network extends through the article.

The non-porous starting thermoplastic article may be contacted with the composition comprising solvent and non-solvent in any suitable manner. For example, the article may be submersed into the liquid composition, the composition may be sprayed on, pipetted on, casted on, inkjetted on, contacted printed on, dropped on, or otherwise applied to the article, the composition maybe vaporized and applied to the article, and the like. It has been found that dipping the article into the liquid composition serves as a convenient and readily accessible method for contacting the article with the composition. It has also been found that microporous structures can readily be generated from the articles at room temperatures, further adding to the convenience. Of course, the temperature may be varied as desired or practicable to achieve a suitable microporous network.

The composition comprising the solvent may be removed from the article in any suitable manner, such as removing the article from the solvent/non-solvent composition source and drying. Drying may be facilitated by increasing temperature, suction, vacuum stripping, or blowing air or nitrogen, or the like.

The pore size of the resulting microporous structure may vary depending on, among other things, the composition of the polymeric material, the birefringence of the material, the solvent and non-solvent used, and the like. It has been found that the average size of the pores generated can be moderately controlled by the solvent composition employed. Average pore sizes generated using the methods described herein, in some embodiments, can range from between 1 micrometer to 500 micrometers. While the mechanism of pore formation is not entirely understood, using an alcohol (e.g. isopropanol or ethanol) as a nonsolvent tends to favor the formation of smaller average pore sizes, and water as a nonsolvent tends to favor formation of larger pore sizes on polystyrene substrates.

The resulting microporous structure that forms from the polymeric article may be an interconnected open cell structure or a non-interconnected open cell structure. Again, while the mechanism is not entirely understood, we have found that higher degrees of orientation (higher birefringence) tends to favor formation of more highly interconnected porous structures. Microscopic examination of the microporous structure may give an indication as to whether the resulting microporous structure is interconnected or non-interconnected. By way of further example, one may employ a liquid wicking test to determine whether the generated porous network is interconnected. If a liquid is blocked from moving across the surface of the microporous structure and is capable of moving though the generated porous network, then the generated porous network is interconnected and has an open cell configuration. Any suitable liquid wicking test may be employed. By way of example, such a test may be performed generally as described in EXAMPLE 5 of copending patent application Ser. No. 13/217,912, filed on the same day as the present application, entitled FLEXIBLE MICROFLUIDIC DEVICE WITH INTERCONNECTED POROUS NETWORK, naming Po Ki Yuen and Michael E. DeRosa as inventors, and having attorney docket no. SP10-234, which application is hereby incorporated herein by reference in its entirety to the extent that it does not conflict with the present disclosure.

In various embodiments, a polymeric article with a patterned microporous structure is fabricated. To produce the patterned article, a mask may be applied to a surface of the article prior to contacting the article with the composition comprising the one or more solvents. Any suitable mask may be used. The mask should prevent the surface of the article from being contacted with the solvent composition, e.g., when submersed in the composition. Additionally, the mask should be readily removable from the article and should not be soluble in the one or more solvents used. In many embodiments, self adhesive tape or other films may be used as a mask. In some embodiments, it may be desirable to mask one entire surface of the article and to pattern mask the opposing surface to produce a desired microporous structure only on one surface of the article.

One convenient way to form a film mask with a desired pattern is to use a desktop digital cutting device, such as described in, for example, P. K. Yuen and V. N. Goral, “Low-cost rapid prototyping of flexible microfluidic devices using a desktop digital craft cutter”, Lab on a Chip, 2010, 10, 384-387. Of course, any other suitable method may be used to cut or produce a mask to a desired pattern.

The pore forming process caused by contacting the polymeric article with the composition comprising the solvent may be ended by any suitable mechanism, such as removing the composition comprising the solvent from the article. The composition may be removed in any suitable manner, such as removing the article from the composition source and drying. Drying may be facilitated by increasing temperature, vacuum stripping, or blowing air or nitrogen, or the like. In embodiments, the article is contacted with a non-solvent composition (e.g., having a Hansen RED for the polymer of about 2.2 or higher) that is miscible with the one or more solvents in the solvent composition to extract the solvent from the article. The non-solvent composition, which may contain extracted solvent composition, may be removed, e.g. by drying.

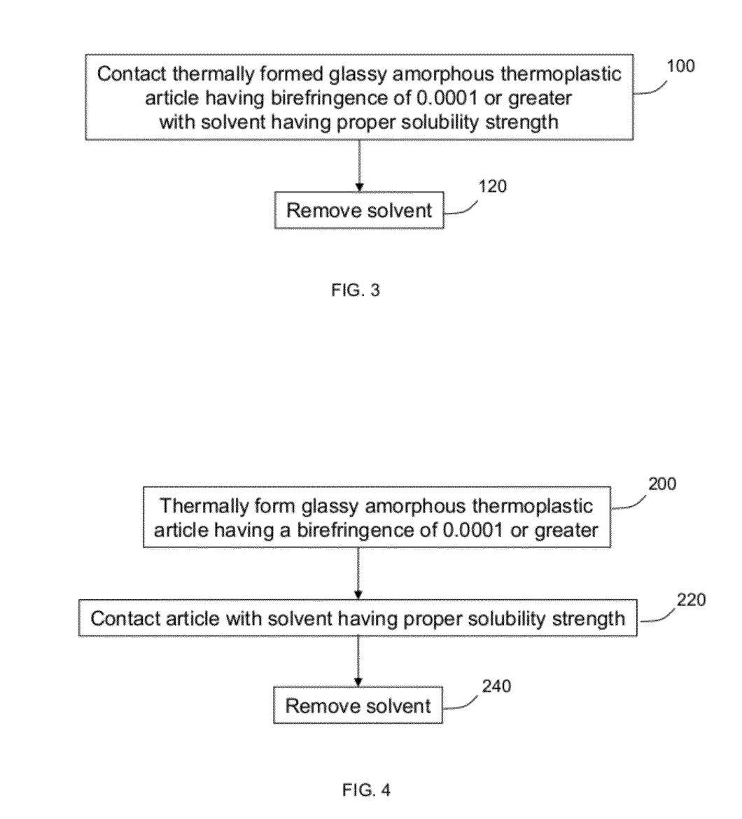

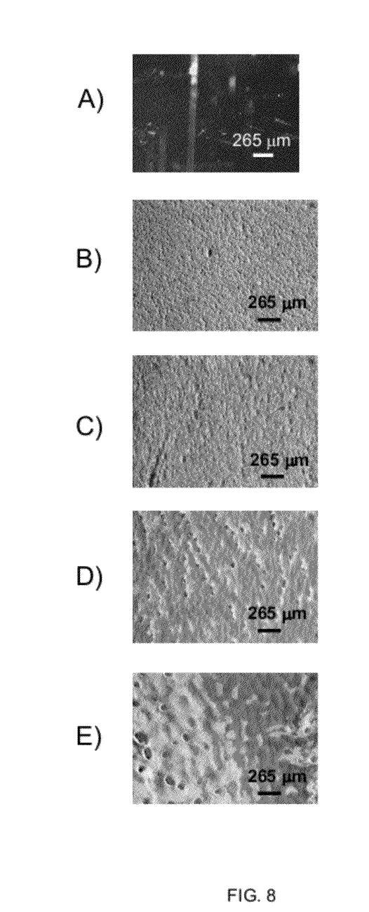

Referring now to FIGS. 3-5, overviews of methods for fabricating thermoplastic articles having microporous structures are shown. As shown in FIG. 3, a thermally formed glassy amorphous thermoplastic article having a birefringence of 0.0001 or greater is contacted with a composition comprising solvent having proper solubility strength (100). If the composition having proper solubility strength has a solubility parameter higher than that of the polymer which it should be sufficient to swell but not dissolve the polymeric article. As discussed above, the composition may also include a non-solvent, and the appropriate ratio of solvent to non-solvent may be used to produce a desired microporous structure from the article. The composition comprising the solvent is removed (120) and the article having a microporous structure on its surface is thus produced. So, for the purposes of this disclosure, proper solubility strength is a solvent or a combination of solvent and non-solvent having a solubility parameter higher then that of the polymer, sufficient to swell but not fully dissolve the polymeric article (see, for example, FIG. 8).

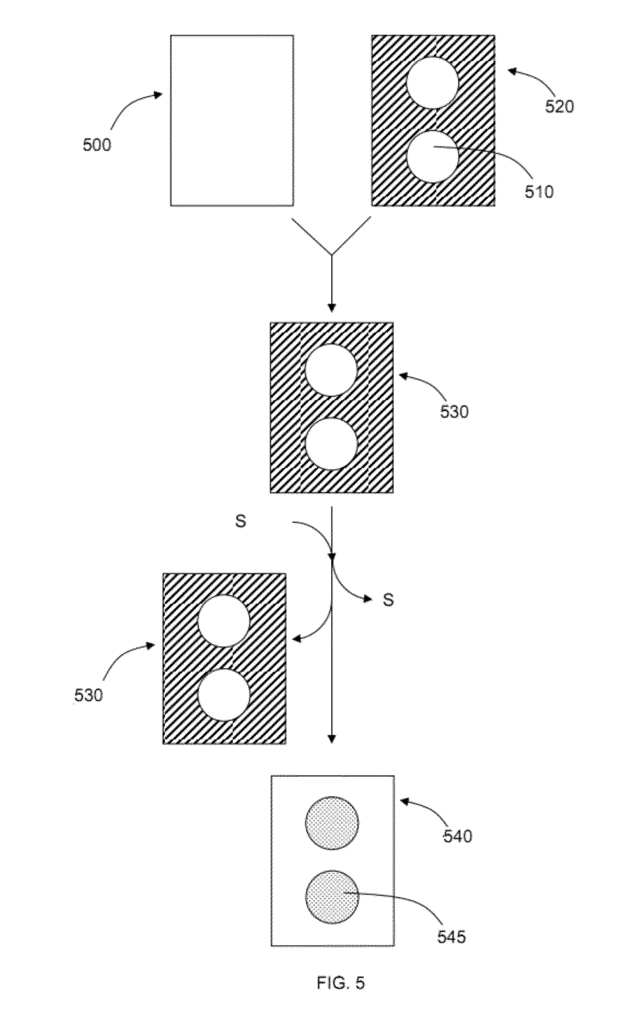

As shown in FIG. 3, the article is provided. As used herein, “provided,” “providing,” “provide,” or the like, in the context of a method as described herein, means purchase, manufacture or otherwise obtain. The method depicted in FIG. 4 includes thermally forming the glassy amorphous thermoplastic article so that it has a birefringence of 0.0001 or greater (200). Thermoplastic polymers can be formed by extrusion, molding or the like. They may be heated above their glass transition temperature and then cooled to form a glassy amorphous structure. Any technique may be used to ensure the desired degree of molecular orientation, such as those discussed above, which include controlling extruding or molding parameters such as injection speed, melt temperature, gate location and size, mold temperature, etc. The thermally formed article is then contacted with an appropriate composition having a solvent (220), and the solvent composition is removed (240), resulting in a thermoplastic article having a microporous structure. The solvent composition may be removed in any suitable manner, such as removing the article from the solvent composition source and drying. Drying may be facilitated by increasing temperature, suction, or blowing air or nitrogen, vacuum stripping, or the like. In embodiments, the article is contacted with a non-solvent composition (e.g., having a Hansen RED for the polymer of about 2.2 or higher) that is miscible with the one or more solvents in the solvent composition to extract the solvent from the article to effectively remove the solvent composition from the article and to arrest the pore forming process.

Referring now to FIG. 5, an example of a method for producing a thermoplastic article having patterned microporous structures is shown in diagrammatic form. First, a mask 500 having patterned openings 510 is placed on a surface of a thermoplastic article having a birefringence of 0.0001 or greater 500 to produce a masked article 530. As indicated above, any suitable mask, such as adhesive tape, may be used. The masked article 530 is then contacted with a composition comprising a solvent (S), the composition is removed, and the mask 520 is removed to produce an article having microporous structured regions 5450. The microporous structured regions 5450 correspond to the unmasked areas, and the non-porous regions correspond to the areas that were masked.

3. Modification of Properties of Microporous Network

Many polymeric articles have hydrophobic surfaces, and rendering the surfaces microporous may increase the hydrophobicity of the article. In some applications of the microporous articles, the increased hydrophobicity may be desirable. However, in other applications, a more hydrophilic surface may be desired. The microporous surfaces generated according to the methods described above may be treated in any suitable manner to increase the hydrophilicity or wettability of the surface. For example, plasma treatment, such as oxygen plasma treatment, may be employed. One suitable method for forming more hydrophilic surface that may be employed is Corning Incorporated's CELLBIND process, e.g. as described in U.S. Pat. No. 6,617,152, which is incorporated herein by reference in its entirety to the extent that it does not conflict with the present disclosure. Other methods for increasing hydrophilicity or wettability of a surface, such as those described in U.S. Pat. No. 4,413,074, which is incorporated herein by reference in its entirety to the extent that it does not conflict with the present disclosure, may be employed. In U.S. Pat. No. 4,413,074 a hydrophobic polymer surface is contacted with a solution containing hydroxyalkyl cellulose and a perfluorocarbon surfactant in water (or a mixture of water and one or more aliphatic alcohols) to form a layer of the solution on the surface. The surface is then heated to form a bond between the cellulose and the surface, rendering the surface more hydrophilic. Of course, any other methods such as UV ozone or arc plasma may be employed to increase the hydrophilicity or wettability of a microporous surface.

In some embodiments, a hydrophobic thermoplastic article having a microporous structure is rendered hydrophilic in a patterned manner. To produce such an article having patterned hydrophilic regions, a mask may be applied to a surface of the article prior to subjecting the microporous structure of the article to the hydrophilic treatment. Any suitable mask may be used. In many cases, the mask may be a mask as described above with regard to producing a patterned microporous structure. For example, the mask may be formed from self adhesive tape or other film. Regardless of composition of the mask, the mask should prevent the underlying surface of the article from being rendered hydrophilic when the sheet is subjected to the hydrophilic treatment. Preferably, the mask is readily removable from the sheet following the treatment.

4. Uses for Thermoplastic Articles Having Microporous Structures

The polymeric articles having microporous structures are described above may be used for any application in which such microporous structures are desired. The articles may form parts of more complex devices. The articles described herein may be used in lateral flow assays, cell culture ware, microfluidic devices, filtration devices, high surface area substrates for chemical reactions, and the like. In many embodiments, the articles produces as described herein are used as, or a part of, a disposable device or components. However, the article may be employed for longer-term use as desired or practical.

One interesting application that lends itself well to the microporous thermoplastic articles described herein is cell culture. Surface porosity can be added to any cell culture article or part formed of a thermoplastic polymer, provided that the particle has a suitably high birefringence (e.g., 0.0001 or greater). For example, surface porosity can be added to multi-well plates, Petri dishes, cell culture flasks or the like made from polystyrene, cyclic olefin copolymers, or the like.

For example, a bottom plate of a multi-well cell culture article may be masked to expose only those areas of the plate that would correspond to the bottom of the wells in the fully assembled device. The masked plate could then be contacted with a composition comprising solvent (the composition having the appropriate solvent strength). The solvent composition may then be removed yielding a plate with patterned microporous regions corresponding to the bottom of the wells. A treatment, such as oxygen plasma treatment may then be applied, if desired, to render the resulting microporous region more hydrophilic to improve cell binding. In some cases, the mask that was used in the process to generate the microporous structures may be left in place and used during the hydrophilic treatment process. The mask may then be removed and the part corresponding to the sidewalls of the wells may then be welded, thermally joined, adhered or otherwise affixed to the bottom plate to produce a multi-well cell culture article having microporous bottom surfaces.

Another example of a process for preparing a cell culture article in accordance with the teachings presented herein is to form a cell culture substrate from a thermoplastic film. The film may be stretched or otherwise formed to have birefringence of 0.0001 or greater. An appropriate solvent or combination of solvent and non-solvent may be used to render the film, or a surface thereof, microporous. The microporous film may be used in a cell culture article as a cell culture substrate. For example, the film may serve as the bottom of a well of Petri dish or a multi-well culture plate.

When thermoformed thermoplastic articles are rendered microporous in accordance with the teachings presented herein, it may be desirable to take certain precautions with the articles due to the built in stress associated with the increased birefringence. For example, if the microporous articles are part of a larger article, it may be desirable to adhere, rather than weld, to the microporous article, as heat associated with welding may cause cracking or crazing or undesired deformation of the microporous article. Another precaution that may be warranted in some circumstances is to transport the articles under controlled conditions. For example, it may not be desirable to allow the article to be carried in an uncontrolled train car traveling through Arizona in peak summer.

As shown in the EXAMPLES, microporous articles having an average pore size of about 50 micrometers or greater tend to allow cells cultured on the articles to be viewed by routine light microscope techniques, while the images of cells on articles with smaller average pores sizes tend to be distorted. It is believed that the surface irregularity caused by the created microporous structures causes light scattering and results in poor image quality via light microscopy. The light scattering may also result in some opacity. In many cases, substrates that allow 50% or more light to be transmitted through the substrate (50% or greater transmittance relative to a non-porous substrate of the same material) provide surfaces on which cells can be suitably observed via standard light microscope techniques. On many substrates that allow less than 50% transmittance, cell images are too distorted to clearly see cell morphology or some cellular structures. Of course, some substrates can allow for more than 50% transmittance but still result in optical distortion (presumably due to light scattering). Water, in many embodiments, is a suitable non-solvent for producing microporous cell culture substrates that allow ready cell observation via a light microscope.

It will be understood that the examples discussed above are only some of the suitable uses of thermoplastic articles having microporous surface regions generated by the processes described herein and that the articles fabricated according to the methods described herein may be used for any suitable purpose in any suitable device or application.

5. Summary of Selected Disclosed Aspects

This disclosure in various aspects describes methods and articles.

In a first aspect a method for fabricating microporous surface on a thermally formed glassy amorphous thermoplastic article is described. The method includes: (i) providing the thermally formed glassy amorphous thermoplastic article, wherein the article has a birefringence of 0.0001 or greater; (ii) contacting a surface of the article with a composition comprising a solvent for the thermoplastic article, wherein the composition has a solubility strength configured to cause swelling of the thermoplastic article without dissolving the thermoplastic article; and (iii) removing the composition from the thermoplastic article. In embodiments of this aspect, the composition comprising the solvent has a relative energy difference from the cyclic olefin copolymer of between 0.5 and 2 (e.g., 0.75-1.6, 0.8-1.5, or 0.85-1.45).

A second aspect is a method of the first aspect, wherein the composition comprises a mixture of the solvent and a non-solvent.

A third aspect is a method of the first of second aspect, wherein the thermoplastic article is formed from polystyrene.

A fourth aspect is a method of the third aspect, wherein the composition comprises a mixture of the solvent and a non-solvent, wherein the solvent is tetrahydrofuran or ethyl acetate and wherein the non-solvent is water or a C1-C4 unsubstituted alcohol.

A fifth aspect is a method of the fourth aspect, wherein the ratio of solvent to non-solvent is from 30/70 to 70/30 on a volume/volume basis.

A sixth aspect is a method of the fifth aspect, wherein the solvent is tetrahydrofuran and the non-solvent is an alcohol selected from the group consisting of ethanol and isopropanol, and wherein the ratio of solvent to non-solvent is from 35/65 to 50/50 on a volume/volume basis.

A seventh aspect is a method of the fifth aspect, wherein the solvent is ethyl acetate and the non-solvent is isopropanol, and wherein the ratio of solvent to non-solvent is from 45/55 to 65/35 on a volume/volume basis.

An eighth aspect is a method of the fifth aspect, wherein the solvent is tetrahydrofuran and the non-solvent is water, and wherein the ratio of solvent to non-solvent is from 45/55 to 65/35 on a volume/volume basis.

A ninth aspect is a method of the first or second aspect, wherein the thermoplastic article is formed from a cyclic olefin copolymer.

A tenth aspect is a method of the ninth aspect, wherein the solvent is methylene chloride.

An eleventh aspect is a method of the tenth aspect, wherein the composition consists essentially of methylene chloride.

A twelfth aspect is an method of the ninth aspect, wherein the composition comprises a mixture the solvent and a non-solvent, wherein the solvent is tetrahydrofuran and the non-solvent is water or a C1-C4 unsubstituted alcohol, and wherein the ratio of the solvent and the non-solvent is from 70/30 to 99/1 on a volume/volume basis.

A thirteenth aspect is a method of the ninth aspect, wherein the composition comprises a mixture the solvent and a non-solvent, wherein the solvent is tetrahydrofuran and the non-solvent is isopropanol, and wherein the ratio of the solvent and the non-solvent is from 75/25 to 90/10 on a volume/volume basis.

A fourteenth aspect is a method of the ninth aspect, wherein the composition comprises a mixture the solvent and a non-solvent, wherein the solvent is tetrahydrofuran and the non-solvent is water, and wherein the ratio of the solvent and the non-solvent is from 90/10 to 98/2 on a volume/volume basis.

A fifteenth aspect is a method of the first or second aspect, wherein the thermoplastic article is formed from a styrene maleic anhydride polymer.

A sixteenth aspect is a method of the fifteenth aspect, wherein the solvent is selected from the group consisting of acetone, tetrahydrofuran, 1,3-dioxolane, methylethyl ketone, toluene, ethyl acetate, N-methylpyrolidone.

A seventeenth aspect is a method of the sixteenth aspect, wherein the composition comprises a solvent and a non-solvent, and wherein the non-solvent is selected from the group consisting of water and a C2-C4 unsubstituted alcohol.

An eighteenth aspect is a method of the seventeenth aspect, wherein the ratio of solvent to non-solvent is from 25/75 to 99/1 on a volume/volume basis.

A nineteenth aspect is a method of any of aspects 1-18, wherein the article has a birefringence of 0.001 or greater.

A twentieth aspect is a cell culture article having a surface for culturing cells, wherein the surfaces consists essentially of a molded polymeric material having a surface for culturing cells, wherein the surface comprises a microporous structure formed from the polymeric material.

A twenty-first aspect is an article of the twentieth aspect, wherein the polymeric material is selected from the group consisting of a cyclic olefin copolymer and a polystyrene.

A twenty-second aspect is an article of the twentieth or twenty-first aspect, wherein at least a portion of the surface is oxygen plasma treated.

A twenty-third aspect is a method for forming a microporous cell culture substrate. The method includes: (i) molding a non-porous cell culture substrate from a thermoplastic polymer such that the non-porous substrate has a birefringence of 0.0001 or greater; (ii) contacting a surface of the non-porous substrate with a composition comprising a solvent for the thermoplastic polymer, wherein the composition is configured to cause swelling of the thermoplastic polymer without dissolving the thermoplastic polymer; and (iii) removing the composition from the surface to yield a cell culture substrate having a microporous region contiguous with the surface. In embodiments of this aspect, the composition comprising the solvent has a relative energy difference from the cyclic olefin copolymer of between 0.5 and 2 (e.g., 0.75-1.6, 0.8-1.5, or 0.85-1.45).

A twenty-fourth aspect is a method of the twenty-third aspect, wherein the composition comprises a mixture of the solvent and a non-solvent.

A twenty-fifth aspect is a method of the twenty-third or twenty-fourth aspect, wherein the thermoplastic polymer is selected from the group consisting of a polystyrene, a cyclic olefin copolymer, and a styrene maleic anhydride polymer.

A twenty-sixth aspect is a method of the twenty-third aspect, wherein the thermoplastic polymer is a polystyrene, wherein the composition comprises a mixture of the solvent and a non-solvent, wherein the solvent is tetrahydrofuran or ethyl acetate and wherein the non-solvent is water or a C1-C4 unsubstituted alcohol.

A twenty-seventh aspect is a method of the twenty-third aspect, wherein the thermoplastic polymer is a cyclic olefin copolymer, wherein the composition consists essentially of methylene chloride or comprises a mixture of the solvent and a non-solvent, wherein the solvent is tetrahydrofuran and the non-solvent is water or a C1-C4 unsubstituted alcohol.

A twenty-eighth aspect is a method of the twenty-third aspect, wherein the thermoplastic polymer is a styrene maleic anhydride polymer, wherein the composition comprises a mixture of the solvent and a non-solvent, wherein the solvent is selected from the group consisting of acetone, tetrahydrofuran, 1,3-dioxolane, methylethyl ketone, toluene, ethyl acetate, N-methylpyrolidone, and wherein the non-solvent is water or a C1-C4 unsubstituted alcohol.

A twenty-ninth aspect is a method of any of aspects 23-28, wherein the substrate has a birefringence of 0.001 or greater.

A thirtieth aspect is a method for fabricating microporous surface on a polystyrene article. The method includes: (i) providing a polystyrene article having a birefringence of 0.0001 or greater; (ii) contacting a surface of the article with a composition comprising a solvent for the polystyrene, wherein the composition is configured to cause swelling of the polystyrene article without dissolving the polystyrene article; and (iii) removing the composition from the polystyrene article. In embodiments of this aspect, the composition comprising the solvent has a relative energy difference from the cyclic olefin copolymer of between 0.5 and 2 (e.g., 0.75-1.6, 0.8-1.5, or 0.85-1.45).

A thirty-first aspect is a method of the thirtieth aspect, wherein the composition comprises a mixture of the solvent and a non-solvent, wherein the solvent is tetrahydrofuran or ethyl acetate and wherein the non-solvent is water or a C1-C4 unsubstituted alcohol.

A thirty-second aspect is a method of the thirty-first aspect, wherein the ratio of solvent to non-solvent is from 30/70 to 70/30 on a volume/volume basis.

A thirty-third aspect is a method of the thirty-second aspect, wherein the solvent is tetrahydrofuran and the non-solvent is an alcohol selected from the group consisting of ethanol and isopropanol, and wherein the ratio of solvent to non-solvent is from 35/65 to 50/50 on a volume/volume basis.

A thirty-fourth aspect is a method of the thirty-second aspect, wherein the solvent is ethyl acetate and the non-solvent is isopropanol, and wherein the ratio of solvent to non-solvent is from 45/55 to 65/35 on a volume/volume basis.

A thirty-fifth aspect is a method of the thirty-second aspect, wherein the solvent is tetrahydrofuran and the non-solvent is water, and wherein the ratio of solvent to non-solvent is from 45/55 to 65/35 on a volume/volume basis.

A thirty-sixth aspect is a method of any of aspects 30-35, wherein the article is a molded article.

A thirty-seventh aspect is a method of any of aspects 30-35, wherein the article is a film.

A thirty-eighth aspect is a method of any of aspects 30-37, wherein the article comprises a cell culture substrate.

A thirty-ninth aspect is a cell culture article comprising a polystyrene article prepared according to the method of the thirty-eighth aspect.

A fortieth aspect is a method for fabricating microporous surface on a cyclic olefin copolymer article. The method includes: (i) providing a cyclic olefin copolymer article having a birefringence of 0.0001 or greater; (ii) contacting a surface of the article with a composition comprising a solvent for the cyclic olefin copolymer, wherein the composition is configured to cause swelling of the cyclic olefin copolymer article without dissolving the cyclic olefin copolymer article; and (iii) removing the composition from the cyclic olefin copolymer article. In embodiments of this aspect, the composition comprising the solvent has a relative energy difference from the cyclic olefin copolymer of between 0.5 and 2 (e.g., 0.75-1.6, 0.8-1.5, or 0.85-1.45).

A forty-first aspect is a method of the fortieth aspect, wherein the solvent is methylene chloride.

A forty-second aspect is a method of the fortieth aspect, wherein the composition consists essentially of methylene chloride.

A forty-third aspect is a method of the fortieth aspect, wherein the composition comprises a mixture the solvent and a non-solvent, wherein the solvent is tetrahydrofuran and the non-solvent is water or a C1-C4 unsubstituted alcohol, and wherein the ratio of the solvent and the non-solvent is from 70/30 to 99/1 on a volume/volume basis.

A forty-fourth aspect is a method of the fortieth aspect, wherein the composition comprises a mixture the solvent and a non-solvent, wherein the solvent is tetrahydrofuran and the non-solvent is isopropanol, and wherein the ratio of the solvent and the non-solvent is from 75/25 to 90/10 on a volume/volume basis.

A forty-fifth aspect is a method of the fortieth aspect, wherein the composition comprises a mixture the solvent and a non-solvent, wherein the solvent is tetrahydrofuran and the non-solvent is water, and wherein the ratio of the solvent and the non-solvent is from 90/10 to 98/2 on a volume/volume basis.

A forty-sixth aspect is a method of any of aspects 40-45, wherein the article is a molded article.

A forty-seventh aspect is a method of any of aspects 40-45, wherein the article is a film.

A forty-eighth aspect is a method of any of aspects 40-47, wherein the article comprises a cell culture substrate.

A forth-ninth aspect is a cell culture article comprising a polystyrene article prepared according to the method of the forty-eighth aspect.

A fiftieth aspect is a cell culture article a microporous substrate suitable for observation of cells cultured on the surface via light microscopy. The substrate is formed from a thermoplastic polymer and has an open cell microporous structure having an average pore size of 50 micrometers or greater.

A fifty-first aspect is a cell culture article according to the fiftieth aspect, wherein the substrate has 50% or greater visible light transmittance.

A fifty-second aspect is a cell culture article according to the fiftieth or fifty-first aspect, wherein the substrate consists essentially of a molded polymeric material.

A fifty-third aspect is a cell culture article according to the fiftieth or fifty-first aspect, wherein the substrate consists essentially of a film.

A fifty-fourth aspect is a cell culture article according to any of aspects 50-53, wherein the substrate comprises a polystyrene or a cyclic olefin copolymer.

A fifty-fifth aspect is a cell culture article according to any of aspects 50-54, wherein at least a portion of the substrate is plasma treated.

A fifty-sixth aspect is a method for forming a microporous cell culture substrate, wherein the substrate allows cells cultured on the substrate to be viewed by routine light microscope techniques. The method comprises: (i) providing a thermally formed non-porous thermoplastic cell culture substrate having a birefringence of 0.0001 or greater; (ii) contacting a surface of the non-porous substrate with a composition comprising a non-solvent and a solvent for the thermoplastic polymer, wherein the composition is configured to cause swelling of the thermoplastic polymer without dissolving the thermoplastic polymer, wherein the non-solvent is water; and (iii) removing the composition from the surface to yield a cell culture substrate having a microporous region contiguous with the surface.

A fifty-seventh aspect is a method according to the fifty-sixth aspect, wherein the substrate has 50% or greater visible light transmittance.

A fifty-eighth aspect is a method according to the fifty-sixth or fifty-seventh aspect, wherein the solvent is tetrahydrofuran.

A fifty-ninth aspect is a method according to the fifty-eighth aspect, wherein the ratio of solvent to non-solvent in the composition is between 45/55 to 98/2 on a volume/volume basis.

A sixtieth aspect is a method according to any of aspects 56-59, wherein the thermoplastic substrate comprises polystyrene or a cyclic olefin copolymer.

A sixty-first aspect is a method according to the sixtieth aspect, wherein the thermoplastic substrate comprises polystyrene, wherein the solvent is tetrahydrofuran, and wherein the ratio of tetrahydrofuran to water is 45/55 to 65/35 on a volume/volume basis.

A sixty-second aspect is a method according to the sixtieth aspect, wherein the thermoplastic substrate comprises a cyclic olefin copolymer, wherein the solvent is tetrahydrofuran, and wherein the ratio of tetrahydrofuran to water is 90/10 to 98/2 on a volume/volume basis.

A sixty-third aspect is a method according to any of aspects 56-62, wherein the thermoplastic substrate is molded.

A sixty-fourth aspect is a method according to any of aspects 56-63, wherein the thermoplastic substrate is a film.

In the following, non-limiting examples are presented, which describe various embodiments of the articles and methods discussed above.

### EXAMPLES

### Example 1

### Effect of Polymer Molecular Orientation on Surface Pore Formation

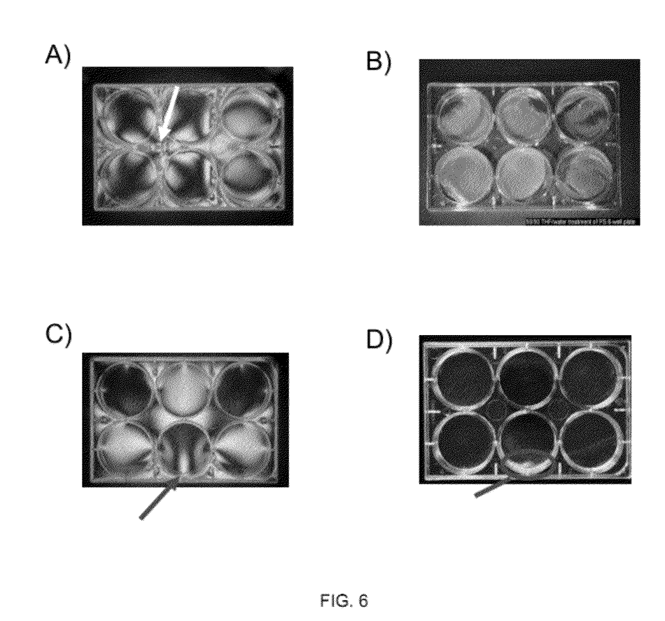

It has been found that, in order to produce a microporous surface from a thermoformed thermoplastic article, the article should be made with at least a minimum amount of molecular orientation. One parameter that can be used to control molecular orientation is the location of the injection gate into the mold to create a high shear zone across the volume of the mold. We demonstrated how the injection gate location can affect molecular orientation in a complex molded 6-well plate. We used two 6-well plates: one is a polystyrene plate that was center gate molded and the other is a cyclic olefin copolymer 6-well plate that was side-gate molded. FIGS. 6A and 6C show cross-polarized images of the two plates molded by different methods: (A) polystyrene; (C) cyclic olefin copolymer. The polystyrene part that was center gate molded (the arrow in FIG. 6A indicates the injection location) has colored fringes (birefringence) across a greater portion of all 6 well bottoms, while the side gate molded cyclic olefin copolymer piece only has a high degree of birefringence in a small location on the bottom of well number 5 which is the middle well in the bottom row (indicated by the arrow in FIG. 6C).

Each plate was solvent treated with a pore-inducing solvent mixture to show the impact of molecular orientation on creating surface porosity. 1 ml of a 40/60 v/v mixture of THF/isopropanol to each well of the polystyrene plate was added. We then added 1 ml of an 80/20 v/v mixture of THF/isopropanol was then added to each well of the TOPAS® plate. After 30 seconds the solvent was removed and the plates were blown dried with nitrogen.

The solvent treatment reveals the impact of residual molding stress and hence molecular orientation on the homogeneity of the surface porosity that is produced after solvent treatment. The plate that was center gate molded has much more coverage of surface porosity over most of the wells (see FIG. 6B, the polystyrene plate). There are some patterns of non-porosity in wells 3 and 6 which are furthest from the injection port location, and as a result have a lower level of shear-induced birefringence. The plate that is side gate molded has porosity only in a small location in well #5 closest to the side injection location where the molecular orientation is highest (see FIG. 6D, the cyclic olefin copolymer plate). This result indicates that in order to get homogeneous coverage of porosity with solvent treatment on molded parts, the residual stress should be elevated and homogeneous over the entire area (or at least exceed the minimum birefringence threshold across the entire area). This could be accomplished by either center gate molding or by using multiple gates to inject molten polymer to increase the level of shear stress.

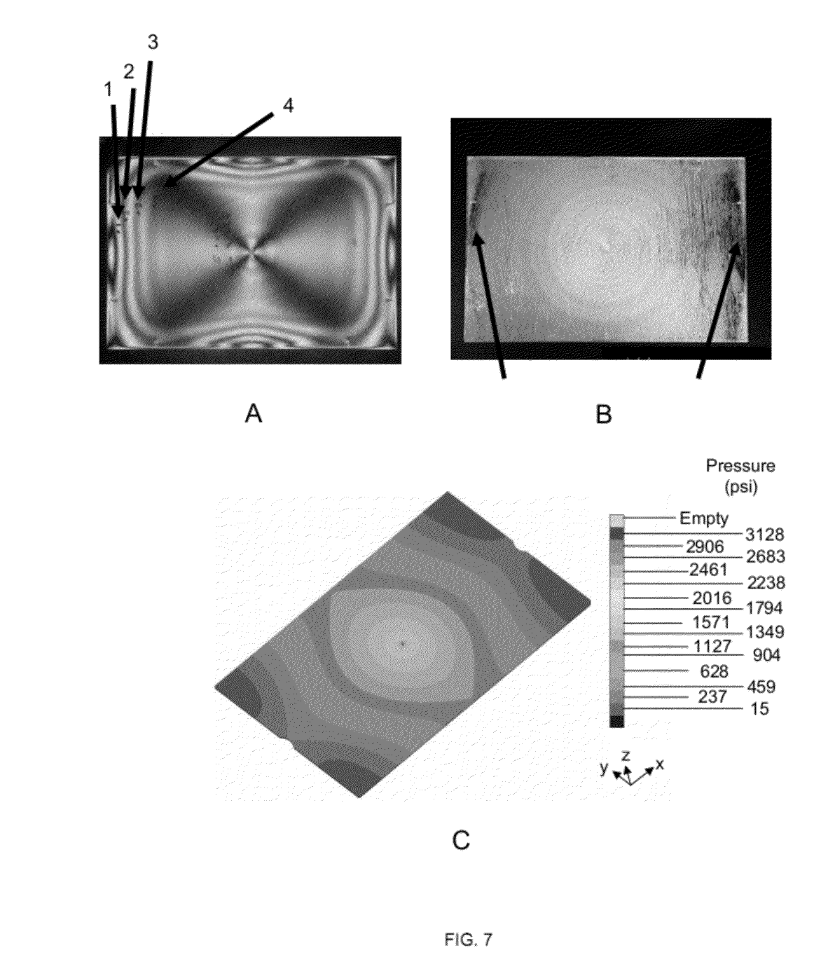

We conducted an experiment to determine the minimum value of birefringence necessary to create solvent induced porosity in polystyrene. We obtained a center gate molded rectangular piece of polystyrene (Dow Styron® 685D). FIG. 7A is an image of the plate between crossed polarizers. The birefringence across the plate fringes was measured and is labeled in FIG. 7A: with the birefringence at 1 being 0.000897; the birefringence at 2 being 0.00179; the birefringence at 3 being 0.00269; and the birefringence at 4 being 0.00359. The plate was dipped in a mixture of 60/40 v/v THF/water mixture for 30 s. The resulting surface porosity created on the piece is shown in FIG. 7B. As can be seen, the regions of lowest birefringence (0.000897) have only a sparse amount of pitting on the surface while the homogeneity of the surface coverage of porosity increases as the birefringence increases toward the center of the plate near the injection location. Therefore, the surface solvent treatment will create the most homogenous coverage of porosity on the surface when the absolute value of the birefringence is greater than a value of 0.0001 and preferably greater than 0.001.

FIG. 7C shows the flow simulation result showing the injection pressure plot from which shear field can be inferred

### Example 2

### Effect of Solvent on Surface Pore Formation

We observed that the solvent type or range of a mixture of solvent with non-solvent produced the desired porous surface coverage. FIGS. 8A-E show how a range of mixtures of tetrahydrofuran (THF) and isopropanol (IPA) affect the surface of polystyrene: (A) 25/75 THF/IPA; (B) 35/65 THF/IPA; (C) 40/60 THF/IPA; (D) 50/50 THF/IPA; and (E) 60/40 THF/IPA. A drop of the solvent mixture was applied to the surface of an injection molded rectangular insert plate made of 685D polystyrene. The solvent was allowed to evaporate before taking images. The results show that below a v/v ratio of THF/IPA of 35/65 there is no effect on the surface texture (see FIG. 8A). In a range between 35/65-50/50 THF/IPA we see that we get open cell surface porosity (see FIGS. 8B-D). Above a 50/50 mixture of these solvents on polystyrene we only get an inhomogeneous softened surface that is white in color with no porosity (see FIG. 8E). Thus, when using the solvent treatment method, the proper range of solvent and non-solvent should be employed to create the desired homogenous porous surface and to create a solvent having the proper solubility.

Repeating similar experiments with different combinations of solvents, non-solvents and polymers, we found that for polystyrene the following mixtures of solvent and non-solvent were effective in producing a desired microporous surface structure: tetrahydrofuran (THF) and isopropanol in a v/v range of 35/65-50/50; THF and ethanol in a v/v range of 35/65-50/50; ethyl acetate and isopropanol in a v/v range of 45/55-65/35; and THF and water in a v/v range of 45/55-65/35. For cyclic olefin copolymer we found that the following mixtures of solvent and non-solvent were effective in producing a desired microporous surface structure: methylene chloride (single solvent); THF and isopropanol in a v/v range of 75/25-90/10; and THF and water in a v/v range of 90/10-98/2.

It will be understood that these are just examples that were found to work successfully and these do not constitute an exhaustive list of solvents, non-solvents, and polymers for which the processes described herein will produce microporous surface structures.

### Example 3

### Direct Solvent Treatment of Molded Part

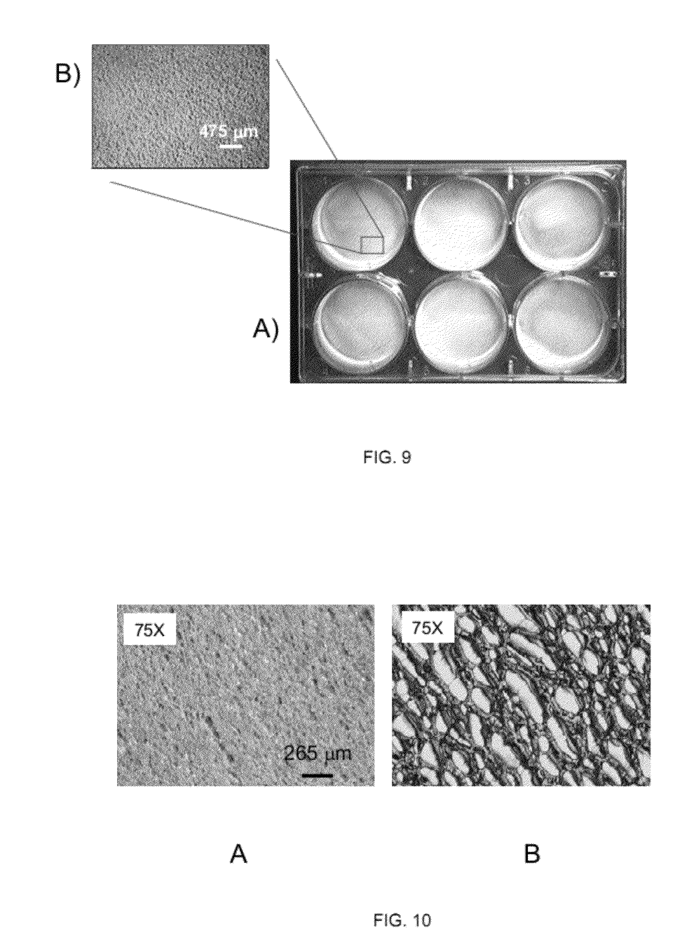

Once the proper solvent or mixture of solvent and non-solvent have been found by screening a surface, a molded part can be solvent treated to make the surface microporous. To demonstrate this approach, we used a Corning Costar® (Corning Incorporated, Corning, N.Y.) molded polystyrene 6-well plate that was center gate molded. We applied 1 ml of a 40/60 v/v mixture of THF/isopropanol to each well. We let the solvent sit for approximately 30 seconds and then removed the residual solvent. The wells were blow dried with nitrogen and then vacuum stripped at 50° C. at 25 inches Hg overnight. The result is shown in FIG. 9.

### Example 4

### Pore Size

We observed that the pore size could be moderately controlled by adjusting the solvent ratio. On molded polystyrene articles we could control the pore size from approximately 30-50 microns up to nearly 300 microns depending on the solvent mixture used. FIGS. 10A-B show optical images of a polystyrene 6-well bottom surface that was treated for 30 s with a 40/60 mixture of THF/IPA (A) and a 50/50 mixture of THF/water (B). The THF/IPA mixture made a fine pore structure with an estimated mean pore diameter of approximately 30-50 microns while the THF/water treated surface made pores that had an estimated size in the range of 100-300 microns.

When using 3 mil thick Trycite® (Dow Chemical, Midland Mich.) polystyrene film we saw a broader range of pore sizes. A piece of Trycite film was dipped in a 40/60 THF/IPA mixture for 20 seconds. The sample was blow dried for approximately 2 min. A second specimen of film was dipped in a 60/40 THF/IPA mixture for 20 seconds and blow dried for approximately 2 min. The specimen dipped in the THF/IPA mixture produced interconnected pores with a mean pore size of approximately 5 microns while the specimen dipped in the THF/water mixture produced non-interconnected pores with a mean size in the range of 100-300 microns.

### Example 5

### Stencil Patterning and Post Surface Treatment of Porous Structure

Using a stenciling process to make patterns of microporous surface regions on the molded article, patterned areas were made on a rectangular plate made of cyclic olefin copolymer. The plate was used as a bottom insert that can be attached to a 96-well holey plate using and adhesive gasket. A flat plate made of COC (Topas®) available from TOPAS® advanced polymers Florence, Ky. grade 8007-X10 was injection molded. The molded article has dimensions of approximately 117 mm long and 76 mm wide, and 1 mm thick. The X10 grade material was chosen because it has the least amount of additives in the resin and no processing lubricant package. In a set of preliminary experiments, it was determined that molding at injection speeds slower than 10 inch/second and using melt temperatures of higher than 230° C. at the nozzle produced parts that did not make homogeneous porous surfaces after solvent treatment. The molding process was therefore developed to produce parts at high shear stress conditions. For the Topas 8007 grade, high shear conditions means fast injection speeds (10 inch/second), a fairly cold melt (210° C. at the nozzle) and a mold temperature less than 65° C.

After molding, a 96-well gasket was applied (purchased from ABgene Ltd.) that has pressure sensitive adhesive on both sides of it. The gasket was used as the mask to pattern spots with a 5 mm diameter that will serve as well bottoms of a 96-well plate. The mylar protection sheet was removed from one side of the gasket and the gasket was adhered to the molded plate. The other side of the gasket has a protective mylar sheet with holes in it to protect the gasket and keep the adhesive on the gasket covered. The entire backside of the plate was protected with standard cellophane tape to prevent it from being contacted by the solvent.

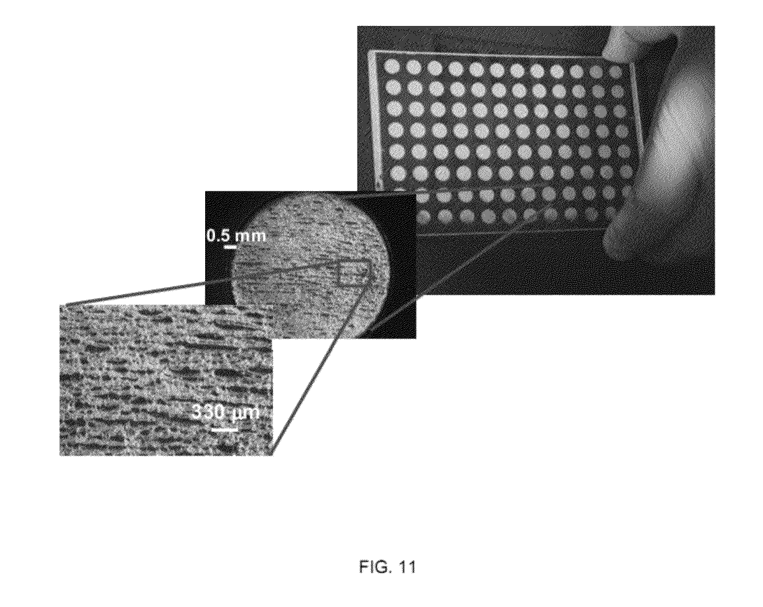

The masked polymer part was immersed in a solvent mixture of 95% THF and 5% water by volume. The part was submerged at room temperature in the mixture for 30 s. After allowing the initial THF to evaporate in a hood for several minutes, the stencil mask and the backside protective tape film was removed. The part was then vacuum stripped in a vacuum oven at 50° C. at 25 in Hg for 5 hours to remove any residual THF. The resulting porous pattern is shown in FIG. 11.

By using a digital craft cutter, more complex stencils can be designed and applied to either one or both sides of the molded part.

### Example 6

### Plasma Treatment

The microporous structures on the patterned polystyrene were made hydrophilic by treating them with oxygen plasma. To demonstrate this we used a piece of microporous polystyrene film that was made by dipping the film into a 40/60 THF/IPA mixture for 20 seconds. After the solvent was removed, we applied tape to one half of the film to mask it from being plasma treated. The masked film was placed in an RF plasma chamber (Model MPS-300; March Instruments, Inc., Concord, Calif., USA) and exposed to oxygen plasma at 40 W for 60 s while oxygen gas was flowing to the chamber. The tape was removed and hydrophilicity of the porous surface was tested by placing a drop of aqueous food coloring dye or water on each side of the plasma treated and untreated film. FIG. 10A shows the result. The side that was plasma treated wicked the food coloring via capillary action rapidly while the untreated side was hydrophobic. The contact angle of the hydrophobic side was measured to be approximately 120 degrees. The plasma treatment to the film was found to be stable for more than 90 days. FIG. 10B shows water wicking on the plasma treated porous surface 90 days after plasma treatment compared to the untreated side which is hydrophobic.

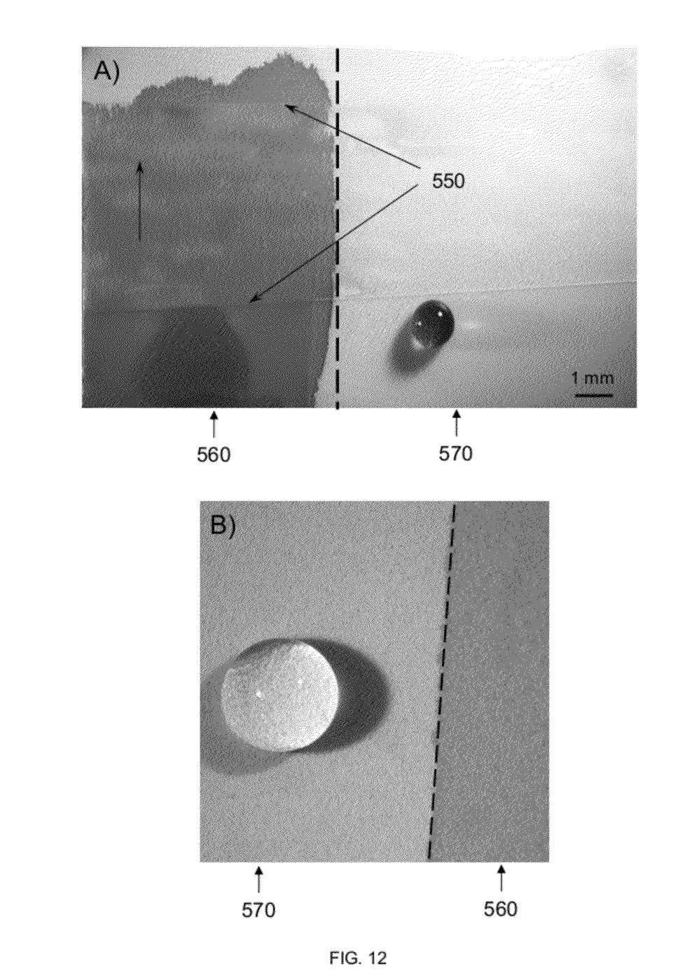

In FIG. 12A, the arrow depicts the direction of wicking. Label number 550 indicates the edges of the transparent tape; 560 indicates oxygen plasma treated side and 570 indicates the side not receiving plasma treatment, with the demarcation between treated and untreated indicated by the dashed line. The microporous structures remained hydrophilic and wicked liquid even more than 90 days after oxygen plasma treatment (FIG. 12B). In FIG. 12B, label number 560 indicates oxygen plasma treated side and 570 indicates the side not receiving plasma treatment, with the demarcation between treated and untreated indicated by the dashed line.

### Example 7

### Production of Cell Culture Articles

Corning Costar polystyrene 6-well plates were rendered microporous. In one plate, we added 1 ml of 50/50 v/v % tetrahydrofuran/water mixture to each well and allowed it to sit for approximately 30 seconds. The microporous structure formed immediately after the solvent mixture contacted the surface of the well bottom. We allowed most of the THF to evaporate into a hood. The remaining water left behind in the well was extracted with a micropipetter and discarded. The wells were then blow dried with nitrogen. In another 6-well plate we added 1 ml of a 40/60 v/v % tetrahydrofuran/isopropanol mixture to each well and allowed the liquid to sit for approximately 30 seconds. The same extraction and blow drying process was used. Both plates were then put into a vacuum oven at 50° C. and 25 inches Hg vacuum overnight to strip any residual THF solvent in the polymer surface. After vacuum stripping, the plates were oxygen plasma treated for 60 s at 40 W in an RF oxygen plasma chamber (Model MPS-300; March Instruments, Inc.) to improve wetting on the surface.

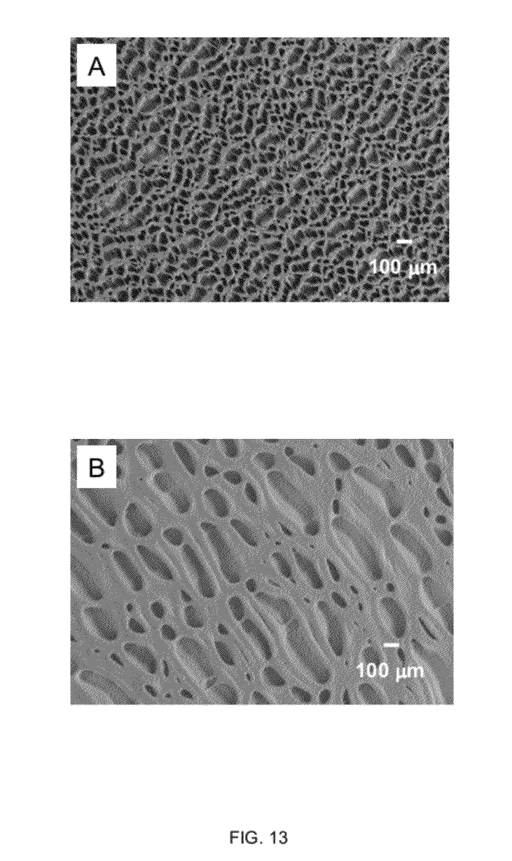

Stereo optical microscope images of the well plates made with each solvent treatment are shown in FIG. 10, where (A) shows the plate treated with the 40/60 mixture of THF/IPA and (B) shows the plate treated with a 50/50 mixture of THF/water. Backscatter SEM images of well bottoms are shown in FIG. 13, further emphasizing the differences in the porous structure generated by the different solvent/nonsolvent mixtures. In FIG. 13, (A) is an image of the THF/IPA treated plate and (B) is an image of the THF/water treated plate. Both images are at 50× magnification.

The THF/IPA mixture made a fine pore structure with a mean pore diameter of approximately 31.4+/−19 microns, while the THF/water treated surface made pores that had a size in the range of 59.7+/−46 microns, as determined by image analysis. Many of the pores made by the THF/water treatment appear oblong in shape and much larger lengthwise than the mean pore size that was calculated. The image analysis method may not be accurately reflecting the anisotropic nature of these pores and therefore the “true” average pore size made by this type of solvent treatment.

The plates with the smaller pores (THF/IPA treated) are opaque and do not readily allow for light microscopic observation of cells cultured on the microporous surface. While not intending to be bound by theory, the opacity of the material itself may be due to some form of microphase separation or crystallization within the polystyrene as a result of the solvent treatment. We observed similar opacity in the material when we used ethanol as the nonsolvent or used another mixture such as ethylacetate and isopropanol. Again, without intending to be bound by theory, it is believed that the irregular surfaces scatter light to an extent that images obtained via a standard light microscope are too distorted to suitably view cell morphology or cellular structures.

However, when we used water as the nonsolvent we found that we could make much larger pores and additionally the scaffold material was transparent (or not opaque) or did not scatter light to an extent to create a large amount of optical distortion, which allows for enhanced ability to view cells growing within the pores (relative to the opaque surfaces).

In addition to using injection molded well plates, we also demonstrated that the same effect could be produced in polystyrene film. Such films can be used as bottom inserts for multi-well plates or as removable insert discs for multiwell plates. We made microporous polystyrene 3 mil films (TRYCITE 1003U, Dow Chemical, Midland, Mich.) by dipping a piece of film in either 50/50 THF/water or 60/40 THF/IPA mixture for 15 s. The solvents were allowed to evaporate in the hood and then the films were vacuum stripped overnight at 50° C. at 25 in Hg to strip any residual THF.

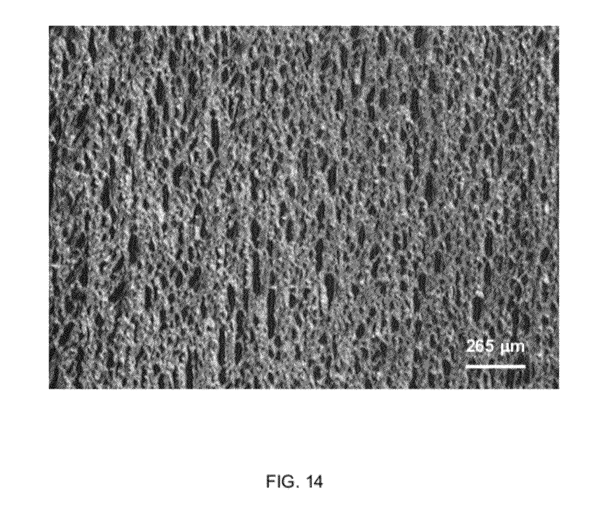

In addition to polystyrene, we also were able to make large semi-transparent microporous surfaces in cyclic olefin copolymer thermoplastics (TOPAS), which surfaces allowed for observation of cells via light microscopy. We used a rectangular bottom insert plate of TOPAS® grade 8007-X10 that was injection molded. We dipped the plate into a mixture of 95/5 THF/water v/v % for 30 s. The resulting porous morphology is shown in the optical image in FIG. 14. Since TOPAS has a different molecular structure and resistance to solvents than polystyrene, we used a different solvent/nonsolvent ratio. We found that we could make large open cell porosity with semi-transparent scaffold structures using a range of THF/water from 80/20 v/v % to 95/5 v/v % THF/water. The best results were achieved using a range of THF/water from 90/10-95/5 v/v %. As with the case with polystyrene, the TOPAS® porous surface could further be treated with oxygen plasma to enhance its wettability.

### Example 8

### Culturing of Cells on Mircroprous Articles

We conducted cell culture experiments to show the advantage for cell culture observation and image acquisition that the large pore transparent or semi-transparent polystyrene matrix has over the smaller pore opaque polystyrene.

A. Human Mesenchymal Stem Cell (hMSC) Culture

hMSCs were purchased from Lonza. Cells were thawed following the manufactory instruction and grew the hMSCs in Lonza's MSCGM™ containing fetal bovine serum, L-glutamine at 37oC with 5% CO2. The passage 4 of hMSC were seeded at 5000 cells per well of 96-well or 150,000 cells per well of 6-well plate. The cells were observed with an inverted light microscope (Zeiss Axiovert 200M) and we captured images using a digital camera (AxioCam MRm) linked to Zeiss Axiovert 200M microscope. The routine cell culture examination was performed with a phase microscope (Zeiss ID03).

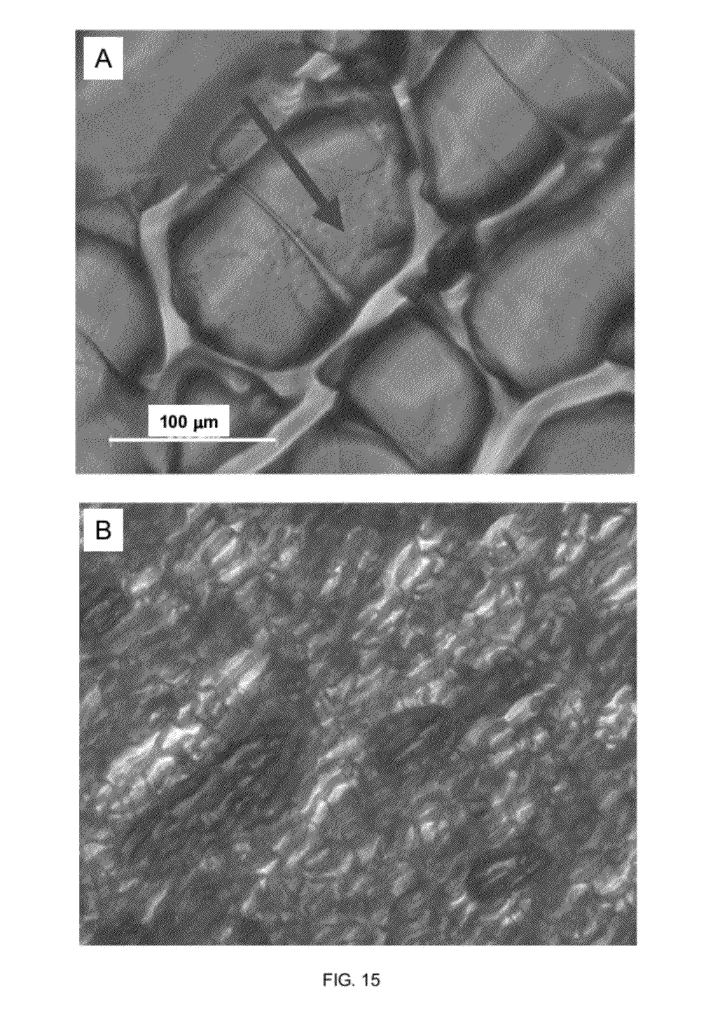

After 2 hours and one overnight of cell culture, the images of cells were captured with the light microscope. The images of cells attached to the microporous polystyrene with the larger pore size (THF/water treatment) were captured 2 hours after seeding the cells (FIG. 15A, the arrow indicates observed cells). The microporous polystyrene with the smaller pore size (THF/IPA treatment) is opaque. Although it was difficult to observe the cells on the microporous polystyrene with small pore size, the images of substrate were still captured after 2 hours of cell culture. No cells could be observed on the microporous polystyrene with the smaller pore size (FIG. 15B).

B. Actin and Nuclei Staining

After one overnight cell culture, the hMSCs were rinsed twice with pre-warmed phosphate-buffered saline (PBS), pH 7.4, and then the samples were fixed in 3.7% formaldehyde solution in PBS for 10 minutes at room temperature (RT). Prior to pemealizing the cells with a solution of 0.1% Triton X-100 in PBS for 5 minutes at RT, the cells were washed two times with PBS.

For actin staining, the stock solution of fluorescent FITC labeled phalloidin (Invitrogen) was diluted into 1 to 50 dilutions in PBS. To reduce nonspecific background staining with these conjugates, 1% bovine serum albumin (BSA) was added to the staining solution. 500 μl FITC-phalloidin dilutions were placed into each well of 6-well plate and incubated for 20 minutes at room temperature. The plates were covered foil paper to shield from light with during the staining.

For nuclei staining, the hMSCs were washed two times with PBS at room before adding 4′-6-Diamidino-2-phenylindole (DAPI) staining solution. The dilution of DAPI (Vector Laboratories) at 1:5 in PBS was added to each well prior to florescent microscopic observation and imaging.

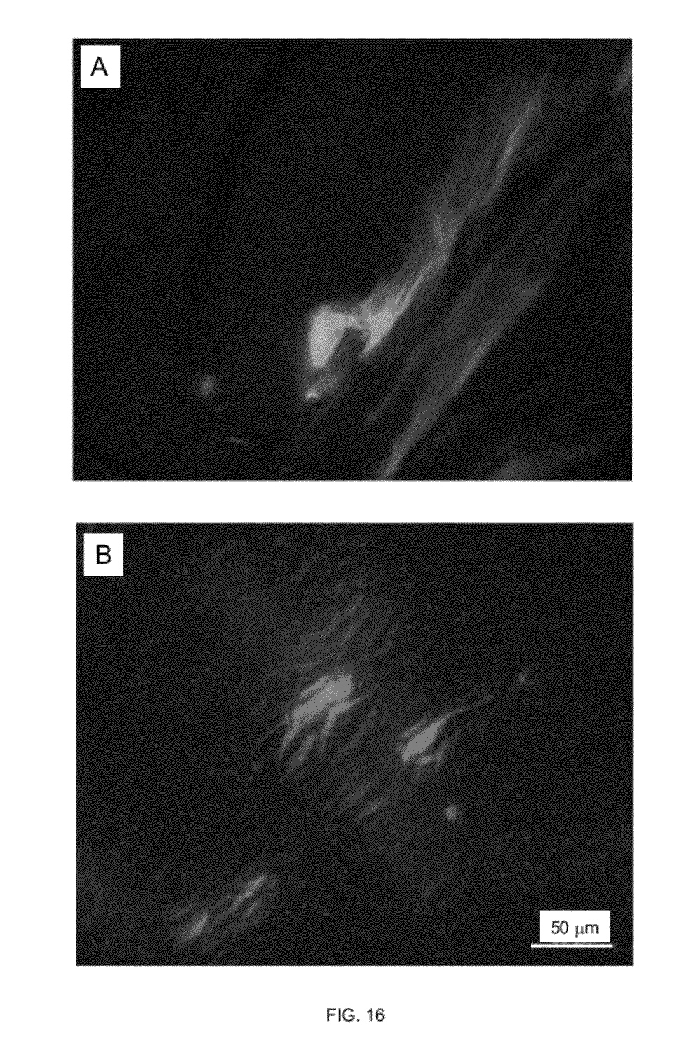

The results showed that the fine structures of fluorescently stained cells can be observed on microporous polystyrene with large pores size (FIG. 16A), but the not on the substrate with small pore size (FIG. 16B). The images were captured under fluorescent microscope using the FITC channel (494 nm/521 nm, excitation/emission) and DAPI channel (358 nm/461 nm, excitation/emission).

C. Cell Attachment Assay

CytoTox 96® Non-Radioactive Cytotoxicity Assay Kit (Promega) was used to test the MC3TC cell culture seeded on polystyrene microporous with large and small pores, 2D polystyrene, and 3D polystyrene inserts (3D Biotek) in 96-well format. The cytosolic enzyme Lactate Dehydrogenase (LDH) is released from the cells using Triton-x-100 (Sigma). The colorimetric measurement provides a non-radioactive method for measuring this LDH activity.

After overnight culture in alpha MEM plus 10% FBS and 1% penn/strep at 37° C. and 5% CO2, MC3T3 cells were washed with PBS, Cells were lysed with 1% of Triton-X-100 in PBS. 50 μl of cell lysate from each well was transferred to a fresh flat bottom 96-well assay plate (Corning) and then mixed with 50 microliter substrate in assay buffer. The reaction was protected from strong direct light by covering the plate with foil paper for 10 minutes at room temperature. 50 microliter of stop solution was added to each well and the assay plate was read the absorbance at 490 nm with Wallec plate reader (Perkin Elmer).

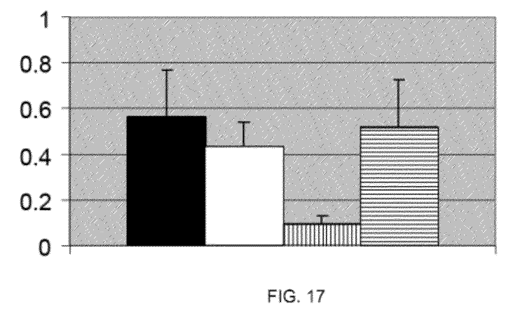

The LDH assay indicated that the cell numbers of MC3T3 attached to microporous polystyrene with large and small was similar to the 2D substrate. The cells attached to the benchmark 3D polystyrene inserts were significantly less than on microporous polystyrene with large and small pores. The benchmark 3D polystyrene inserts were also significantly less than on 2D polystyrene (FIG. 17). In FIG. 17, the y-axis reflects the OD at 490 nm all indication of cell attachment. The black bar represents the larger pore polystyrene article (THF/water), the white bar indicates the smaller pore polystyrene article (THF/IPA), the vertically striped bar represents the 3D Biotek substrate and the horizontally striped bar represents the 2D (non-porous) polystyrene surface.

The results presented herein indicate that optically transparent or semi-transparent microporous cell culture articles can be made in accordance with the teachings presented herein. The transparent or semi-transparent articles allow for observation of cultured cells using standard light microscope techniques. It has been found that articles having an average pore size of about 50 micrometers or more allow for observation by light microscope, while microporous culture articles with smaller pore sizes tend to be too opaque to allow for useful light microscopic observation.

### Example 9

### Hansen Solubility Parameters for Solvents that Form Microporous Surfaces on Polystyrene Articles

As described in more detail in co-pending U.S. patent application Ser. No. 13/217,818, entitled MICROPOROUS THERMOPLASTIC SHEETS, having attorney docket no. SP11-197, naming Michael DeRosa, Todd Upton, and Ying Zhang as inventors, and filed on the same date herewith, we performed testing to determine which solvents or mixtures of solvents and non-solvents formed microporous surfaces on polystyrene articles and determined the Hansen RED values of those solvents and solvent/non-solvent mixtures that were effective in pore formation. A brief overview of those studies is presented herein.

Briefly, Hansen solubility parameters for solvent mixtures that form microporous surfaces on a molded polystyrene cell culture plate, which had a gradient of birefringence values across the surface (with a significant portion being greater than 0.001), were determined in the following manner. First, a range of known solvents and non-solvents for polystyrene were tested on the surface of a molded polystyrene cell culture plate. 50-100 microliters of each test solvent and non-solvent were pipetted onto the surface of the polystyrene at room temperature. Observations were made under a microscope to see if the solvent dissolved the surface within a 2 min time period. Once a range of solvents and non-solvents were tested (see Table 1), the Hansen parameters, δP and δH, were plotted against each other for each test solvent. This type of two dimensional plot shows one cross section of the total three dimensional polystyrene solubility sphere.

TABLE 1
Solvents and non-solvents used to determine Hansen Solubility Parameters

Solvents
1,1,1-Trichloroethane
Methylene Dichloride (Dichloromethane)
N-Methyl-2-Pyrrolidone
Ethyl Acetate
Dimethylformamide
n-Butyl Acetate
Chlorobenzene
Cyclohexanone
Isoamyl Acetate
1,3-Dioxolane
Toluene
Acetone
1,1-Dichloroethane
Tetrahydrofuran
Diethyl Ether
Methyl Ethyl Ketone
Non solvents
Cyclohexane
2-Propanol
Ethyl Lactate
Methanol
Dimethyl Sulfoxide
Glycerol
Water
Propylene Carbonate
1-Butanol
Ethanol

Using HSPiP software (Hansen Solubility Parameters in Practice, v.3.1) a fit of the data was calculated to determine the center coordinates of the polystyrene sphere and the solubility radius Rₒ. Data analysis using HSPiP software found the parameters to be δ_{D}=16.98, δ_{P}=6.76 and δ_{H}=4.83 with Rₒ=6.4. 50-100 microliters of solvent/nonsolvent mixtures including tetrahydrofuran/water, tetrahydrofuran/isopropanol, tetrahydrofuran/propylene carbonate, ethylacetate/isopropanol, toluene/dimethyl sulfoxide, acetone/isopropanol, and 1,3 dioxolane/water were pipetted onto the polymer surface allowed to sit for 60 seconds then blow dried. The resulting surface features were observed under a microscope.

HSPiP software was used to determine the Hansen solubility parameters of the solvent/nonsolvent mixtures with the v/v % ranges shown in Table 2. The solubility parameters of the mixtures were plotted against the known solvent and non-solvent values determined earlier. A solubility boundary having a radius Rₒ=6.4 was determined. It was also found that the solubility parameter range of the solvent/non-solvent mixtures that formed microporous surfaces have RED values in the range of 0.88-1.41.

TABLE 2
Solvents with appropriate RED values to form microporous
polystyrene

| Solvent/Non-solvent mixture | Range v/v % |
|---|---|
| Tetrahydrofuran/water | 50/50-65/35 |
| Tetrahydrofuran/isopropanol | 35/65-45/55 |
| Tetrahydrofuran/propylene carbonate | 37/63-50/50 |
| Ethylacetate/isopropanol | 60/40-70/30 |
| Toluene/dimethyl sulfoxide | 25/75-30/70 |
| Acetone/isopropanol | 70/30-80/20 |
| 1,3 Dioxolane/water | 60/40-80/20 |

While the polymeric articles tested in this example were molded cell culture articles, the Hansen solubility parameters should be representative of other polymeric articles.

Thus, embodiments of MICROPOROUS CELL CULTURE SUBSTRATE are disclosed. One skilled in the art will appreciate that the cell culture apparatuses and methods described herein can be practiced with embodiments other than those disclosed. The disclosed embodiments are presented for purposes of illustration and not limitation.

What is claimed is:

1. A method for forming a microporous cell culture substrate, comprising:
molding a non-porous cell culture substrate from a thermoplastic polymer such that the non-porous substrate has a birefringence of 0.0001 or greater;
contacting a surface of the non-porous substrate with a composition comprising a solvent for the thermoplastic polymer, wherein the composition is configured to cause swelling of the thermoplastic polymer without dissolving the thermoplastic polymer; and
removing the composition from the surface to yield a cell culture substrate having a microporous region contiguous with the surface.

2. A method according to claim 1, wherein the composition comprising the solvent has a relative energy difference from the polymer of between 0.5 and 2.

3. A method according to claim 1, wherein the composition comprising the solvent has a relative energy difference from the polymer of between 0.75 and 1.6.

4. A method according to clam 1, wherein the composition comprises a mixture of the solvent and a non-solvent.

5. A method according to claim 1, wherein the thermoplastic polymer is selected from the group consisting of a polystyrene, a cyclic olefin copolymer, and a styrene maleic anhydride polymer.

6. A method according to claim 1, wherein the thermoplastic polymer is a styrene maleic anhydride polymer, wherein the composition comprises a mixture of the solvent and a non-solvent, wherein the solvent is selected from the group consisting of acetone, tetrahydrofuran, 1,3-dioxolane, methylethyl ketone, toluene, ethyl acetate, N-methylpyrolidone, and wherein the non-solvent is water or a C1-C4 unsubstituted alcohol.

7. A method according to claim 1, wherein the non-porous substrate has a birefringence of 0.001 or greater.

8. A cell culture article having a surface for culturing cells, wherein the surface consists essentially of a molded polymeric material having a surface for culturing cells, wherein the surface comprises a microporous structure formed from the polymeric material.

9. A cell culture article having a microporous substrate suitable for observation of cells cultured on the surface via light microscopy, wherein the substrate is formed from thermoplastic polymer and comprises an open cell microporous structure having an average pore size of 50 micrometers or greater.

10. A cell culture article according to claim 9, wherein the substrate has 50% or greater visible light transmittance.

11. A cell culture article according to claim 9, wherein the substrate consists essentially of a molded thermoplastic polymeric material.

12. A cell culture article according to claim 9, wherein the substrate consists essentially of a film.

13. A cell culture article according to any of claims 9, wherein the substrate comprises a polystyrene or a cyclic olefin copolymer.

14. A method for forming a microporous cell culture substrate, wherein the substrate allows cells cultured on the substrate to be viewed by routine light microscope techniques, the method comprising:
providing a thermally formed non-porous thermoplastic cell culture substrate having a birefringence of 0.0001 or greater;
contacting a surface of the non-porous substrate with a composition comprising a non-solvent and a solvent for the thermoplastic polymer, wherein the composition is configured to cause swelling of the thermoplastic polymer without dissolving the thermoplastic polymer, wherein the non-solvent is water; and
removing the composition from the surface to yield a cell culture substrate having a microporous region contiguous with the surface,
wherein the resulting microporous region has an average pore size of 50 micrometers or greater.

15. A method according to claim 14, wherein the substrate has 50% or greater visible light transmittance.

16. A method according to claim 14, wherein the solvent is tetrahydrofuran.

17. A method according to claim 16, wherein the ratio of solvent to non-solvent in the composition is between 45/55 to 98/2 on a volume/volume basis.

18. A method according to claim 14, wherein the thermoplastic substrate comprises polystyrene or a cyclic olefin copolymer.

19. A method according to claim 14, wherein the composition comprising the solvent has a relative energy difference from the polystyrene of between 0.5 and 2.

20. A method according to claim 14, wherein the composition comprising the solvent has a relative energy difference from the polystyrene of between 0.75 and 1.6.
